(19) United States
(12) Patent Application Publication
(10) Pub. No.: US 2026/0114815 A1
(43) Pub. Date: Apr. 30, 2026

(54) NEUROLOGICAL STATE, DISEASE, DYSFUNCTION, OR INJURY IDENTIFICATION SYSTEMS AND DEVICES

(71) Applicant: BrainScope Company, Inc., Rockville, MD (US)

(72) Inventors: Drew Aaron Linsley, Lakeville, MA (US); Leslie S. Prichep, New York, NY (US); Aditya Ramchandra Dalvi, Broomfield, CO (US)

(21) Appl. No.: 19/368,767

(22) Filed: Oct. 24, 2025

Related U.S. Application Data
(60) Provisional application No. 63/711,557, filed on Oct. 24, 2024.
(60) Provisional application No. 63/711,551, filed on Oct. 24, 2024.
(60) Provisional application No. 63/822,607, filed on Jun. 12, 2025.

(51) Int. Cl. A61B 5/00; A61B 5/372; G06N 20/00; G16H 50/20

(52) U.S. Cl. CPC A61B 5/7267; A61B 5/372; A61B 5/4082; A61B 5/4088; A61B 5/4094; A61B 5/4803; G06N 20/00; G16H 50/20

(57) ABSTRACT

Subject measurement systems can generate indications of neurological state, disease, dysfunction, or injury using a machine learning model. The machine learning model can include at least one encoding model and a sequential model pre-trained to perform language processing tasks. The machine learning model can further include a classifier configured to output classifications. The at least one encoding model, sequential model, and at least one decoding model can be jointly trained to predict timeseries output, thereby adapting the pre-trained sequential model for use with neurologically relevant input domains, such as medical images, EEG data, evoked response data, speech data, or the like. The at least one encoding model, sequential model, and classifier can be jointly trained to output indications of neurological state, disease, dysfunction, or injury. A subject measurement system can then generate such indications using patient data and the at least one encoding model, sequential model, and classifier.

### CROSS-REFERENCE TO RELATED APPLICATIONS

This application claims benefit of priority to U.S. Provisional Patent Application No. 63/711,557, filed Oct. 24, 2024, U.S. Provisional Patent Application No. 63/711,551, filed Oct. 24, 2024, and U.S. Provisional Patent Application No. 63/822,607, filed Jun. 12, 2025. Each of these applications are incorporated herein by reference in their entireties.

### TECHNICAL FIELD

Systems for identification of neurological state, disease, dysfunction, or injury using image, textual, speech, or timeseries data. In particular, identification of neurological state, disease, dysfunction, or injury using pre-trained language models adapted for the processing of image, textual, speech, or timeseries data.

### BACKGROUND

Conventional systems for identification of neurological state, disease, dysfunction, or injury can be difficult to develop, improve or implement in the clinical environment. Such systems may use features, parameters, or configurations described in the scientific or clinical literature or reflecting expert knowledge or know-how. Developing such systems may therefore require extensive input from human experts. Once developed, such systems may only accept inputs in specific formats. For example, a medical record of a subject may include notes indicating that the subject exhibited signs of confusion. Such notes may be relevant to identification of the patient as being concussed. But a conventional system configured to diagnose concussion based on EEG timeseries data may be unable to directly use such notes. Furthermore, the output of a conventional system may be difficult to interpret, or such a system may be inflexibly configured to perform a particular analysis.

Conventional systems for identification of neurological state, disease, dysfunction, or injury can be difficult to develop or improve. Such systems may use features, parameters, or configurations reflecting expert knowledge. Developing such systems may therefore require extensive input from human experts. Once developed, such systems may only accept inputs in specific formats. For example, a medical record of a subject may include notes indicating that the subject exhibited signs of confusion. Such notes may be relevant to identification of the patient as being concussed. But a conventional system configured to diagnose concussion based on EEG timeseries data may be unable to use such notes. Furthermore, the output of a conventional system may be difficult to understand, or such a system may be inflexibly configured to perform a particular analysis.

### SUMMARY

The disclosed embodiments include subject measurement systems that generate indications of neurological state, disease, dysfunction, or injury using a machine learning model. The machine learning model can include at least one encoding model and a sequential model pre-trained to perform language processing tasks. The machine learning model can further include a classifier configured to output classifications. The at least one encoding model, sequential model, and at least one decoding model can be jointly trained to predict timeseries output, thereby adapting the pre-trained sequential model for use with neurologically relevant input domains, such as medical images, EEG data, evoked response data, speech data, or the like. In some embodiments, the at least one encoding model, sequential model, and classifier can be jointly trained to output indications of neurological state, disease, dysfunction, or injury. In some embodiments, the disclosed subject measurement system can generate such indications using patient data and the jointly trained at least one encoding model, sequential model, and classifier.

The disclosed embodiments include a subject measurement system. The subject measurement system can include at least one processor and at least one non-transitory, computer-readable medium. The at least one non-transitory, computer-readable medium can include instructions that, when executed by the at least one processor, cause the subject monitoring system to perform operations. The operations can include obtaining an electroencephalographic (EEG) dataset for a subject. The operations can include applying the EEG dataset to a tokenizing model to generate a first token. The operations can include applying the first token to a sequential machine learning model to generate output features. The tokenizing model, the sequential machine learning model, and a de-tokenizing model can be jointly trained to generate an output EEG dataset from an input EEG dataset. The operations can include applying a classifier input including the output features to a classifier to generate an indication of a neurological state, disease, dysfunction, or injury. The operations can include providing the indication of the neurological state, disease, dysfunction, or injury to inform treatment of the subject.

The disclosed embodiments include a method for training a subject measurement system. The method can include an operation of training a first combined machine learning model using an electroencephalographic (EEG) pre-training dataset to generate an output sequence of multi-channel EEG observations from an input sequence of multi-channel EEG observations. The EEG pre-training dataset can include sets of sequential multi-channel EEG observations. The first combined machine learning model can include: a tokenizing model configured to generate a first token from the input sequence, a sequential machine learning model configured to generate a second token from the first token, and a de-tokenizing model configured to generate the output sequence from the second token. Generation of the second token can include generation of output features. The method can include an operation of training a second combined machine learning model using an EEG training dataset to generate an indication of a neurological state, disease, dysfunction, or injury from an input sequence of multi-channel EEG observations. The EEG training dataset can include labeled sets of sequential multi-channel EEG observations, the label for each set indicating a neurological state, disease, dysfunction, or injury. The second combined machine learning model can include: the tokenizing model, the sequential machine learning model, and a classifier configured to generate the indication using a classifier input including the output features.

The disclosed embodiments include a non-transitory computer-readable medium containing instructions that, when executed by at least one processor of a subject measurement system, cause a subject measurement system to perform operations. The operations can include obtaining an electroencephalographic (EEG) dataset for a subject. The operations can include applying the EEG dataset to a tokenizing model to generate a first token. The operations can include applying the first token to a sequential machine learning model to generate output features. The tokenizing model, the sequential machine learning model, and a de-tokenizing model can be jointly trained to generate an output EEG dataset from an input EEG dataset. The operations can include applying the output features to a classifier to generate an indication of a neurological state, disease, dysfunction, or injury. The operations can include providing the indication of the neurological state, disease, dysfunction, or injury to inform treatment of the subject.

The disclosed embodiments include another subject measurement system. The subject measurement system can include at least one processor and at least one non-transitory, computer-readable medium containing instructions. When executed by the at least one processor, the instructions can cause the subject monitoring system to perform operations. The operations can include generating a first token by applying a subject electroencephalographic (EEG) dataset to at least one tokenizing model. The operations can include generating a second token corresponding to a subject textual or image input. The operations can include generating a subject EEG or textual output using the first token and the second token, generation including applying the first token and the second token to a sequential machine learning model, wherein the at least one tokenizing model, the sequential machine learning model, and at least one de-tokenizing model are jointly trained to generate an output training EEG or textual dataset from an input EEG or textual dataset. The operations can include providing the second EEG data or textual output.

The disclosed embodiments include another method for training a subject measurement system. The operations can include training a first combined machine learning model using an electroencephalographic (EEG) and textual pre-training dataset to generate an output sequence of multi-channel EEG observations and textual values from an input sequence of multi-channel EEG observations and textual or image input data. The EEG and textual pre-training dataset can include sequential multi-channel EEG observations for at least one subject, textual or image input data including instructions for processing the sequential multi-channel EEG observations, and output textual data including indications of neurological states, diseases, dysfunctions, or injuries associated with the multi-channel EEG observations. The first combined machine learning model can include: a tokenizing model configured to generate a first token from the input sequence, a sequential machine learning model configured to generate a second token from the first token, and a de-tokenizing model configured to generate the output sequence from the second token. Training the first combined machine learning model can include updating at least one of the tokenizing model or the detokenizing model based on a comparison of the input sequence and the output sequence.

The disclosed embodiments include another non-transitory computer-readable medium containing instructions. When executed by at least one processor of a subject measurement system, the instructions can cause the subject measurement system to perform operations. The operations can include generating a first token by applying a subject electroencephalographic (EEG) dataset to a tokenizing model. The operations can include generating a second token corresponding to a subject textual input. The operations can include generating a subject EEG or textual output using the first token and the second token, generation including applying the first token and the second token to a sequential machine learning model. The tokenizing model, the sequential machine learning model, and a de-tokenizing model can be jointly trained to generate an output training EEG or textual dataset from an input EEG or textual dataset. The operations can include providing the second EEG data or textual output.

The disclosed embodiments include another subject measurement system. The subject measurement system can include at least one processor and at least one non-transitory, computer-readable medium containing. When executed by the at least one processor, the instructions can cause the subject measurement system to perform operations. The operations can include generating a classifier or textual output using at least one timeseries dataset acquired from a subject and a second dataset concerning the subject, a second modality of the second dataset differing from at least one first modality of the at least one timeseries dataset. Generation can include generating at least one first token, at least in part by applying the at least one timeseries dataset to at least one corresponding tokenizing model, generating a second token using the second dataset, and generating a classifier or textual output at least in part by applying the at least one first token and the second token to a sequential machine learning model. The at least one corresponding tokenizing model, the sequential machine learning model, and at least one de-tokenizing model can be jointly trained to generate at least one predicted output timeseries dataset in the at least one first modality from at least one input timeseries dataset in the at least one first modality. The operations can include providing the classifier or textual output.

The disclosed embodiments include another training system. The training system can include at least one processor and at least one non-transitory, computer-readable medium containing instructions. When executed by the at least one processor, the instructions can cause the training system to perform operations. The operations can include training at least one tokenizing model and at least one de-tokenizing model to generate at least one predicted output timeseries dataset in at least one first modality from at least one first input timeseries dataset in the at least one first modality using a sequential machine learning model. Training the at least one tokenizing model and the at least one de-tokenizing model can include generating at least one first token, at least in part by applying the at least one first input timeseries dataset to the at least one tokenizing model, generating a first output sequence, at least in part by applying a first input sequence including the at least one first token to the sequential machine learning model, extracting at least one first output token from the first output sequence, and generating the least one predicted output timeseries dataset, at least in part by applying the at least one first output token to the at least one de-tokenizing model. The operations can further include storing the at least one trained tokenizing model and the at least one trained de-tokenizing model.

The foregoing general description and the following detailed description are exemplary and explanatory only and are not restrictive of the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute part of this disclosure, together with the description, illustrate and serve to explain the principles of various example embodiments.

FIG. 1 depicts an exemplary subject measuring system, in accordance with disclosed embodiments.

FIG. 2A depicts a training method for an exemplary combined machine learning model, in accordance with disclosed embodiments.

FIG. 2B depicts a classification method suitable for training or inference using components of the exemplary combined machine learning model of FIG. 2A, in accordance with disclosed embodiments.

FIG. 3 depicts inputs, outputs, and components of an exemplary multimodal combined machine learning model suitable for training and inference, in accordance with disclosed embodiments.

FIG. 4 depicts a graphical user interface for a subject measuring system, in accordance with disclosed embodiments.

FIGS. 5A to 5C depict exemplary comparisons between actual EEG outputs and EEG outputs generated using a combined machine learning model, in accordance with disclosed embodiments.

FIG. 6 depicts encoding of subject age information into output tokens generated by a combined model trained consistent with disclosed embodiments.

FIG. 7 depicts encoding of subject epileptic status into output tokens generated by a combined model trained consistent with disclosed embodiments.

FIG. 8 depicts a relationship between subject age and subject epileptic status, as encoded into output tokens generated by a combined model trained consistent with disclosed embodiments.

FIGS. 9A and 9B depict the importance to a combined model trained consistent with disclosed embodiments of portions of EEG timeseries data in determining average subject values for epileptic and non-epileptic patients.

FIG. 10 depicts an exemplary schematic of a device for acquiring and processing brain electric signals, consistent with disclosed embodiments.

FIG. 11 depicts an exemplary placement of the device of FIG. 10 on the head of a patient, consistent with disclosed embodiments.

### DESCRIPTION OF EMBODIMENTS

The following detailed description refers to the accompanying drawings. Wherever possible, the same reference numbers are used in the drawings and the following description to refer to the same or similar parts. While several illustrative embodiments are described herein, modifications, adaptations, and other implementations are possible. For example, substitutions, additions, or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

As described herein, a computing system can “obtain” information (e.g., data, instructions, or the like). Unless described otherwise, “obtaining” the information can include retrieving, receiving, transcribing, or creating the information. In some instances, the computing system can retrieve or receive the information in whole or in part from a user interface of the computer system or at least one other computer system. In some instances, creating the information can include retrieving or receiving other information usable to create the information. This other information can be retrieved or received in whole or in part from a user interface of the computer system or at least one other computer system. Unless described otherwise, obtaining information from another system can include obtaining the information directly (e.g., one computing system retrieving or receiving the information from another computing system across a communication link or network) or indirectly (e.g., one computing system retrieving or receiving the information directly from an intermediary computing system that retrieved or received the information directly from another computing system).

Subject measuring systems consistent with disclosed embodiments can enable identification of neurological state, disease, dysfunction, or injury of a subject using a combined machine learning model. The combined machine learning model can include a sequential model pre-trained for language processing tasks and additional tokenizing and detokenizing models trained to adapt the combined machine learning model to processing other data. In some embodiments, the combined machine learning model can be configured to process timeseries data, such as EEG or speech data. In some embodiments, the combined machine learning model can be configured to process additional input data sources, such as image data, textual medical data, or textual instructions. In some embodiments, the combined machine learning model can be initially trained to output predicted timeseries data given input timeseries data. Components of the combined machine learning model can then be trained using labeled training data to output classifications, given the input timeseries data (and optionally additional input data sources). In some embodiments, the combined machine learning model can be trained using labeled training data to output classifications and predicted timeseries data.

As appreciated by the inventors, the inclusion of the pre-trained sequential model improves the computational and storage efficiency of the combined machine learning model. As understood in the art, the performance of machine learning models typically improves with increasing model size. However, training large machine learning models can be compute- and memory-intensive. As appreciated by the inventors, despite being pre-trained to perform language processing tasks, the pre-trained sequential model can be adapted (using tokenizing and detokenizing models) to process timeseries data such as EEG or speech data. Multiple input data sources can, in some embodiments, be translated into the internal vocabulary to the pre-trained sequential model. This architecture can therefore avoid the compute and memory required to train from scratch a comparable sequential model. The tokenizing models can further reduce compute and memory requirements by encoding many timeseries data samples into fewer sequential model input tokens. As the sequential model may be the largest component of the combined machine learning model, reducing the number of input tokens generated for an amount of input timeseries data can reduce the compute and memory required to process the input timeseries data. Furthermore, this reduction in the number of input tokens generated for an amount of input timeseries data can reduce the response time to generate an output when the model is used in a clinical environment.

As appreciated by the inventors, subject measuring systems consistent with disclosed embodiments improve upon conventional diagnostic systems, at least by reducing or eliminating the need for human-encoded parameters, increasing system flexibility and configurability, and increasing system interpretability. For example, conventional diagnostic systems may require human-coded model parameters (e.g., preprocessor configurations, features extracted from input data, classifier configurations, or the like). But the subject measuring systems consistent with disclosed embodiments can learn the features or configurations predictive of neurological state, disease, dysfunction, or injury. The disclosed systems and methods may therefore reduce dependence on human experts in developing subject measuring systems, making such systems easier and faster to develop and transition to clinical practice. As an additional example, subject measuring systems consistent with disclosed embodiments can benefit from a natural language interface provided by the sequential model. A user can provide textual input to the sequential model ranging from questions or instructions to additional data. For example, the user can prompt a subject measuring system to provide an analysis of EEG data, provide an estimate of the patient's sex, age, cognitive assessment results, or the like. The combined machine learning model can condition processing of timeseries data on this textual input. In some embodiments, the combined machine learning model can be configured to provide a textual output. This textual output can describe the subject or explain the reasoning behind the output indications of neurological state, disease, dysfunction, or injury. As may be appreciated, conventional diagnostic systems can lack such a natural language interface, and therefore lack the configurability and transparency provided by such an interface.

Subject measuring systems consistent with disclosed embodiments can enable neural twinning. Such neural twinning can be used to enrich trials with patients having desired neural characteristics. For example, a compound may appear promising as a treatment for Parkinson disease. But other forms of parkinsonism may not be expected to respond to this compound. A clinical trial that includes both patients with Parkinson Disease and (inadvertently) other forms of parkinsonism may underestimate the effectiveness of the compound in treating Parkinson disease. Subject measuring systems consistent with disclosed embodiments can therefore be used to screen candidates for inclusion in the clinical trial. Only patients identified by such systems as having Parkinson Disease may be included in the clinical trial, thereby limiting the clinical trial to patients more likely to benefit and ensuring a more accurate assessment of compound effectiveness.

As may be appreciated, subject measuring systems consistent with disclosed embodiments can enable similar treatment decisions at an individual patient level. For example, such systems can be used to identify a potential cause for a patient reporting generic or unspecific neurological symptoms. A clinician may then suggest appropriate treatment based on the identified cause (e.g., anti-Parkinson drugs when the patient is identified as having Parkinson Disease). Similarly, subject measuring systems consistent with disclosed embodiments can be used to facilitate early intervention. For example, such systems may enable a clinician to diagnose a neurological state, disease, dysfunction, or injury in a subject earlier than might otherwise be possible. The subject may then receive appropriate treatment. For example, such systems may enable earlier diagnosis of Alzheimer's disease, which may enable earlier treatment with anti-amyloid therapies. As may be appreciated, to the extent that such therapies are protective, rather than restorative, patients may benefit greatly from earlier treatment. Likewise, such systems may impact treatment choice by confirming diagnosis, allowing for more aggressive and earlier interventions.

Subject measuring systems consistent with disclosed embodiments can be used to track the effectiveness of treatment or recovery. For example, the recovery of a subject identified as being concussed may be tracked over time using subject measuring systems consistent with disclosed embodiments. Such tracking can enable a more precise determination of when the subject can return to school, athletic activities, or the like. As an additional example, the effect of compound on a subject can be similarly tracked over time, enabling a clinician to determine the effectiveness of the treatment regime. As may be appreciated, systems consistent with disclosed embodiments may be especially suited for such tracking, as they may be less invasive and less costly than other means of measuring treatment effectiveness, such as biomarker assays or medical imaging.

Subject measuring systems consistent with disclosed embodiments can be configured to produce synthetic patient data representative of a patient having a set of desired patient characteristics (e.g., age, disease status, sex, clinical assessment score, EEG profile, or the like). For example, textual input can be used to specify the desired patient characteristics, and a detokenizing model can generate the desired synthetic patient data. Such synthetic data can be used to train other models or test clinical protocols without the privacy concerns arising from storing, transferring, or accessing large collections of protected patient data.

Subject measuring systems consistent with disclosed embodiments can be used to investigate the causes of neurological states, diseases, or dysfunctions. As described herein, such systems can include information about the input data that most contributed to the classification of a subject as having a particular neurological state, disease, or dysfunction. Such information may be used to identify the biological structures or processes characteristics of the neurological state, disease, or dysfunction. Identification of these biological structures or processes can assist in the identification of causes for, or treatments of, of the neurological state, disease, or dysfunction.

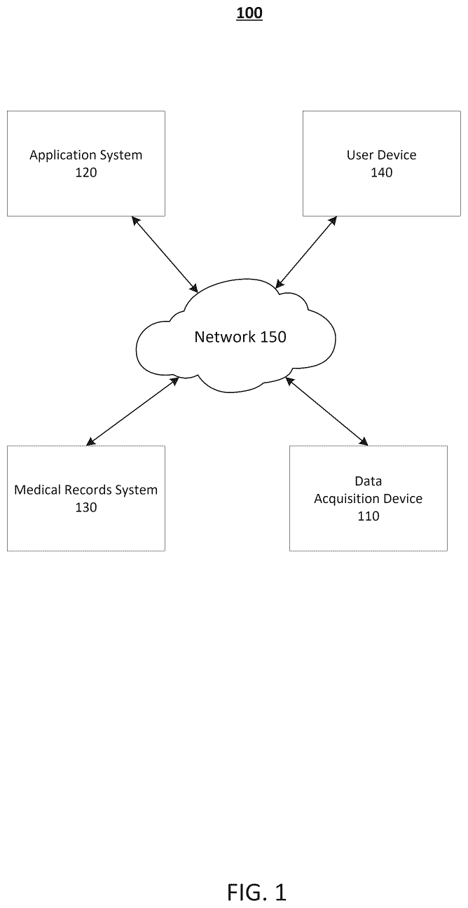

FIG. 1 depicts an exemplary subject measuring system 100, in accordance with disclosed embodiments. Subject measuring system 100 can enable training and deployment of a combined machine learning model useable for identifying neurological state, disease, dysfunction, or injury of a subject. As described herein, the combined machine learning model can include a sequential model pre-trained for language processing tasks. The sequential model can be combined with additional tokenizing, detokenizing, and classifying models that adapt the sequential model for processing other data, such as image data or EEG or speech timeseries data. Such an approach can leverage existing sequential language processing models that might otherwise be too resource-intensive to train.

Furthermore, in some embodiments, the combined machine learning model can support natural language inputs and outputs. For example, as described herein, instructions configuring the processing of input data can be provided as textual inputs to the sequential model. In some embodiments, the sequential model can provide textual outputs indicating the neurological state, disease, dysfunction, or injury of a subject, or additional information concerning the input data.

In some embodiments, subject measuring system 100 can include data acquisition device 110. Data acquisition device 110 can be configured to generate EEG timeseries data by acquiring and processing brain electrical signals. In some embodiments, data acquisition device 110 can be configured to provide the EEG timeseries data to another component of subject measuring system 100, such as application system 120 or medical records system 130. In some embodiments, data acquisition device 110 can be configured to obtain a trained machine learning model (e.g., from application system 120, or the like) and optionally medical data (e.g., from application system 120 or medical records system 130). In some embodiments, data acquisition device 110 can be configured with a user interface. The user interface can enable a user to provide textual input, as described herein, to the machine learning model. In some embodiments, data acquisition device 110 can use the obtained trained machine learning model, EEG timeseries data, and optionally obtained medical data or user input to identify neurological state, disease, dysfunction, or injury in a subject.

In some embodiments, subject measuring system 100 can include an application system 120. Application system 120 can be a computing device, such as a workstation, mainframe, computing cluster, or cloud computing platform configured to support the disclosed systems and methods. For convenience of description, application system 120 is described as performing both training and inference using the combined machine learning model. However, the disclosed embodiments are not so limited. In practice, training may be performed on a computing system with an architecture suitable for training machine learning models (e.g., a computing cluster having significant compute and memory resources connected by high-capacity, high-speed network connections). In some embodiments, the trained machine learning model may then be deployed to a separate computing system with an architecture suitable for responding to user queries (e.g., one or more application servers connected to one or more web servers and one or more back-end systems, or another suitable architecture). In some embodiments, the trained machine learning model may then be deployed to device for clinical use (e.g., data acquisition device 110, or the like).

In some embodiments, application system 120 can obtain medical data, as described herein, from medical records system 130. In some embodiments, application system 120 can obtain this medical data for use in training. For example, application system 120 can create a training dataset using at least some of this medical data (e.g., speech data or EEG data, image data, and textual data describing physiological measurements, clinical pathology test results, clinical data, demographic information, or the like). In some embodiments, application system 120 can be configured to use the medical data for generating labeled training data. For example, when a medical record for a subject includes a diagnosis of “Parkinsons”, then application system 120 can create a training datum including EEG or speech data for that subject and the ground-truth label “Parkinsons”. In some embodiments, application system 120 can obtain this medical data for use in inference. For example, application system 120 can obtain medical record data for a particular subject. In some embodiments, application system 120 can combine this data with data acquired from data acquisition device 110. In some embodiments, application system 120 can be configured with a user interface. The user interface can enable a user to provide textual input, as described herein, to a machine learning model hosted on the application system 120. In some embodiments, the user interface can enable display of an indication of a neurological state, disease, dysfunction, or injury of a subject.

In some embodiments, subject measuring system 100 can include medical records system 130. Medical records system 130 can be a computing device, such as a workstation, mainframe, computing cluster, or cloud computing platform configured to support the disclosed systems and methods. Medical records system 130 can be configured to host an electronic health record system. The electronic health record system can be configured to store patient information, such as medical history, diagnoses, medications, treatment plans, immunization dates, allergies, and laboratory test results. It can also be configured to maintain records of patient interactions, clinical notes, and data from diagnostic devices.

In some embodiments, the electronic health record system can be configured to store medical data concerning a subject. The medical data can include imaging data, such as computed tomography scan data, magnetic resonance imaging, ultrasound data, or the like. The medical data can include speech data of the subject talking (e.g., raw audio data in formats such as WAV, AIFF, or the like; decoded versions of lossy or lossless compressed audio data, such as FLAC, MP3, MPEG-4 ALS, or the like). The medical data can include electroencephalographic (EEG) data, electrocardiographic (ECG) data, electromyographic (EMG) data, electroneurographic (ENG) data, electrooculographic (EOG) data, peripheral electroneurogram data, evoked potential time series data, or the like. The medical data can include physiological measurements such as heart rate variability data, pulse oxygenation data, respiration data, body temperature, or the like. The medical data can include clinical pathology test results, such as blood or fluid biomarker data (e.g., tau or amyloid biomarker measurements, or the like), genetic or genomic descriptor data (e.g., Apo-E variant status, or the like), or the like. The medical data can include clinical data, such as patient cognitive assessment results (e.g., Mini-Mental State Exam (MMSE), Clinical Dementia Rating (CDR), Alzheimer's disease co-operative study activities of daily living (ADCS-ADL), or the like) clinical observations (e.g., “Patient does not have frontal release signs and engages in conversation easily and fluently.”). The medical data can include demographic information (e.g., patient sex, age, height, weight, educational level, or the like), or the like.

In some embodiments, subject measuring system 100 can include user device 140. User device 140 can be a mobile computing device, such as a smartwatch, smartphone, tablet computer, laptop, or the like. In some embodiments, user device 140 can be a desktop or workstation. In some embodiments, user device 140 can be configured to provide a user interface that enables a user to communicate with other components of subject measuring system 100. For example, the user can provide instructions to another component of subject measuring system 100 (e.g., application system 120, data acquisition device 110, or the like). In some embodiments, the user can provide textual input to a machine learning model hosted on the other component of subject measuring system 100. As described herein, such textual input can include input data or instructions for the machine learning model. In some embodiments, user device 140 can be configured to receive (e.g., from application system 120, data acquisition device 110, or the like) an indication of a neurological state, disease, dysfunction, or injury of a subject.

Network 105 can facilitate communications between the other components of subject measuring system 100, consistent with disclosed embodiments. Network 105 can include one or more networks, including a TCP/IP network (e.g., the Internet), a wired Wide Area Network (WAN), a wired Local Area Network (LAN), a wireless WAN (e.g., WiMAX), a wireless LAN (e.g., IEEE 802.11, etc.), a mesh network, a mobile/cellular network, an enterprise or private data network, a storage area network, a virtual private network using a public network, a nearfield communications network (e.g., a Bluetooth link, an infrared link, etc.), or another type of communications network. The disclosed embodiments are not limited to embodiments in which communications between components of subject measuring system 100 occur over a particular type of network.

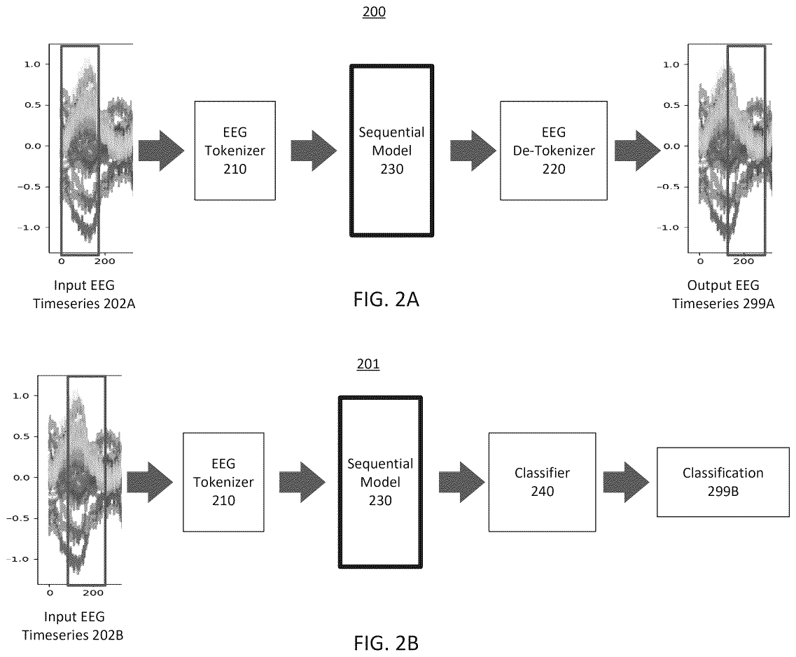

FIG. 2A depicts a training method 200 for an exemplary combined machine learning model, in accordance with disclosed embodiments. For convenience of explanation, method 200 is described as being performed by an application system (e.g., application system 120, or the like) using training data acquired from a patient data acquisition device (e.g., data acquisition device 110, or the like). Furthermore, method 200 is depicted as being performed on EEG data. However, the disclosed embodiments are not so limited. For example, method 200 could be performed entirely on a patient data acquisition device. As an additional example, method 200 could be performed using any suitable timeseries data (e.g., other electrical biosignals, speech data, or the like) or sequential data (e.g., 2D slices of 3D image data, or the like).

Consistent with disclosed embodiments, the combined machine learning model can include a tokenizing model, a sequential machine learning model, and a de-tokenizing model. In some embodiments, the sequential machine learning model can be a large language model pre-trained to perform natural language processing tasks. The sequential machine learning model can be configured to accept sequences of tokens as input and provide sequences of tokens as output. The tokenizing and detokenizing models can be jointly trained with the sequential learning model to adapt the overall combined machine learning model to (in this example) EEG processing tasks, in accordance with disclosed embodiments.

As appreciated by the inventors, the pre-trained large language model may incorporate representations of causality and temporal relationships present in human language. These existing representations can be re-purposed for use in performing EEG processing tasks. Furthermore, while the performance of the combined machine learning model can increase with increasing model size, the tokenizing and detokenizing models may be far smaller than the sequential learning model. Accordingly, training a combined machine learning model that includes a pre-trained large language model may require vastly less memory and computational resources than ab initio training of a similarly sized (and therefore similarly performant) model.

Consistent with disclosed embodiments, the combined machine learning model can be configured to accept timeseries data input and output timeseries data. The input and output time series data may be in the same modality (e.g., EEG, speech data, or the like). The combined machine learning model can be trained to predict a future portion of the input timeseries data. This predicted future portion of the input timeseries data can be compared to the actual future portion of the input timeseries data. Thus, the input timeseries data can serve as the ground truth for unsupervised learning. As described herein, portions of the trained combined machine learning model can then be used to generate a classifier for generating indications neurological state, disease, dysfunction, or injury.

In an operation of method 200, the application system can obtain input EEG timeseries data acquired from a subject (e.g., input EEG timeseries 201A), consistent with disclosed embodiments. The input EEG timeseries data can be obtained from a data acquisition device (e.g., patient data acquisition device 110). As described herein, this data can be obtained directly from the data acquisition device or indirectly from an intermediate system (e.g., a database storing such EEG data for one or more patients). The data acquisition device can obtain the input EEG timeseries data from patient sensors (e.g., patient sensor(s) 110B, or the like) or using signals obtained from such patient sensors. The input EEG timeseries data can include multiple streams of EEG data (e.g., corresponding to different pairs of electrodes). In some embodiments, the EEG timeseries data can be in the time domain (e.g., measured voltage values over time). In some embodiments, the EEG data can be in the frequency domain (e.g., spectrogram data, or the like). The disclosed embodiments are not limited to any particular sampling frequency or temporal resolution of the input EEG timeseries data. For example, the EEG timeseries data can be sampled at (or in the case of frequency domain data, can be generated using timeseries data sampled at) a rate between 60 and 1000 Hz, or higher. The disclosed embodiments are not limited to a particular duration of the input EEG timeseries data. In some embodiments, the input EEG timeseries can include (or in the case of frequency domain, correspond to) at least 10 ms of EEG signals. For example, the input EEG timeseries can include between 10 ms and 1000 seconds of input data, or more. In some embodiments, the input EEG timeseries can be obtained as it is acquired (e.g., by patient data acquisition device 110, or the like).

In an operation of method 200, the application system can generate an input token by applying a portion of the obtained input EEG timeseries data to a tokenizing model (e.g., EEG tokenizing model 210), consistent with disclosed embodiments. The portion can include a number of samples. The disclosed embodiments are not limited to a particular number of such samples. In some embodiments, the portion can include samples corresponding to a duration of between 10 and 100 ms, or longer.

The tokenizing model can be a machine learning model. In some embodiments, the tokenizing model can be neural network, such as a feed-forward neural network. As described herein, the tokenizing model can be trained and configured to adapt the overall combined machine learning model to the performance of EEG processing tasks. The input of the tokenizing model can be suitable for the EEG processing task. For example, the input of the tokenizing model can be vectors or matrices of amplitude values (e.g., time-domain signal measurements from multiple channels, frequency domain measurements for multiple frequency values for multiple channels, or the like).

In some embodiments, the output of the tokenizing model can be adapted to a language processing task. For example, in such embodiments, the tokenizing model can be configured to output a token included within the internal vocabulary of the sequential model. As may be appreciated, such a token can be represented by a one-hot encoding vector with the dimension of the internal vocabulary of the sequential model.

In some embodiments, the tokenizing model can be configured to output a token in a different vocabulary. The application system can then convert this token into a token in the internal vocabulary of the sequential model. For example, a numerical output could be parsed into a string of tokens (e.g., the value 9951.2 could be parsed into the tokens corresponding to “995”, “1”, “.”, and “2”).

As appreciated by the inventors, phenomena of interest in the time series data may occur at far slower timescales than the sampling rate of the input timeseries data. Accordingly, the tokenizing model can be configured to accept as input a first number of timeseries samples (e.g., the portion of the obtained input EEG timeseries data) and output a lesser number of tokens (e.g., accept ten input samples and output one token, or the like). As may be appreciated, this can reduce the number of tokens input to the sequential model. As the sequential model may be the most memory and computationally intensive part of the combined machine learning model, reducing the number of tokens input to the sequential model can reduce the memory and compute required analyze to the input EEG time series. Furthermore, the tokenizing model can shape the input to the sequential model, thereby adapting the sequential model to the performance of EEG processing tasks.

In some embodiments, the tokenizing model can be configured to contract 2-64 samples, or more, sampled with a sampling period of 1 to 50 ms (or in the case of frequency data, corresponding to 1 to 50 ms of EEG data) into a single token.

In an operation of method 200, the application system can generate an output token by applying the input token to sequential model 230, consistent with disclosed embodiments. Sequential model 230 can be a sequential neural network model. In some embodiments, sequential model 230 can be an attention-based model (e.g., a transformer-based model, or the like), a state-space model, or a recurrent neural network. In some embodiments, the sequential neural network model can be configured to generate a new output token using a new input token and a prior model state or output token. In some embodiments, the sequential neural network model can be configured to sequentially add new input tokens to a context window, or slide a context window along a sequence of input tokens. The disclosed embodiments are not limited to any particular architecture. In some embodiments, sequential model 230 can be an implementation of a MAMBA, LLAMA, or GWEN architectures. As described herein, sequential model 230 can be a pretrained language model. This model could include an internal vocabulary suitable for language processing tasks. For example, this internal vocabulary could include tokens corresponding to characters (e.g., ascii characters, unicode characters, or the like), words, subword fragments (e.g., “ing”), or other combinations of characters encountered in the corpus of documents used to create internal vocabulary (e.g., “.SetModel”, “fft”, “_name”, “(“@”, or similar frequently observed combinations of characters).

As may be appreciated, generation of the output token by sequential model 230 can include multiple intermediate processing steps. As a non-limiting example, sequential model 230 can include components, such as hidden layers, or states. As the sequential model processes a sequence of input tokens, the values of these components can change. In some embodiments, the application system can generate output features based on the values of these components. For example, when the sequential model is a recurrent neural network including a hidden state vector, the output features can be or dependent upon values of the hidden state vector. As described herein, components of the combined machine learning model can be used with a classifier to generate an indication of a neurological state, disease, dysfunction, or injury. In some embodiments, the output features can provide the input to the classifier. In some embodiments, the output features can be the output token(s) generated by the sequential model in response to application of the input token.

In an operation of method 200, the application system can generate a portion of predicted EEG timeseries output by applying the output token generated by sequential model 230 to a de-tokenizing model (e.g., EEG De-Tokenizer 220), consistent with disclosed embodiments. The de-tokenizing model can be a machine learning model. In some embodiments, the de-tokenizing model can be neural network, such as a feed forward neural network. As described herein, the de-tokenizing model can be trained and configured to adapt the overall combined machine learning model to the performance of EEG processing tasks.

In some embodiments, the input of the de-tokenizing model can be adapted to a language processing task. For example, the de-tokenizing model can be configured to accept as input a token included within the internal vocabulary of the sequential model. As may be appreciated, such a token can be represented by a one-hot encoding vector with the dimension of the internal vocabulary of the sequential model.

In some embodiments, the de-tokenizing model can be configured to accept as input a token in a vocabulary suitable for the EEG processing task. The application system can convert a token (or sequence of tokens) in the internal vocabulary of the sequential model into a token in this other vocabulary. For example, a string of tokens could be parsed into a numerical output (e.g., the tokens corresponding to “995”, “1”, “.”, and “2” could be parsed into the numerical value 9951.2).

Consistent with disclosed embodiments, the output of the de-tokenizing model can be suitable for the EEG processing task. For example, the output of the de-tokenizing model can be vectors or matrices of amplitude values (e.g., time-domain signal measurements from multiple channels, frequency domain measurements for multiple frequency values for multiple channels, or the like).

In some embodiments, the de-tokenizing model can be configured to expand a single token into a portion of output EEG data including 2-64 samples, or more (or, in some embodiments, the number of samples may differ). These output samples may be deemed to have a sampling period of 1 to 50 ms (or in the case of frequency data, corresponding to 1 to 50 ms of EEG data). In some embodiments, the sampling period of these output samples can be the same as the sampling period of the input samples in the input EEG timeseries (or, in some embodiments, the input and output sampling periods may differ).

As described herein, the tokenizing model can generate a first number of output tokens from a second number of input samples. The de-tokenizing model can generate a third number of output samples from a fourth number of input tokens. In some embodiments, the first and third numbers may be the same number (or, in some embodiments, they may be different numbers). In some embodiments, the second and fourth numbers can be the same number (or, in some embodiments, they may be different numbers).

As described herein, the combined machine learning model can be trained to generate a portion of output EEG data (e.g., a portion of output EEG timeseries 299A) using a portion of input EEG data (e.g., a portion of input EEG timeseries 202A). The portion of output EEG data may be intended to match a subsequent portion of the input EEG data. For example, given times t_{0<}t_{1<}t_{2<}t₃, the combined machine learning model can generate a predicted version of the input EEG data from time t₁ to time t₂ based on a portion of the input EEG data from time t₀ to time t₁. As may be appreciated, the disclosed embodiments are not limited to predicting an adjacent time interval. The combined machine learning model can be trained to predict overlapping time intervals (e.g., predicting the input EEG data from time t₁ to time t₃ based on a portion of the input EEG data from time t₀ to time t₂) or non-contiguous time intervals (e.g., predicting the input EEG data from time t₂ to time t₃ based on a portion of the input EEG data from time t₀ to time t₁).

Consistent with disclosed embodiments, the application system can train the combined machine learning model using an obtained training database of EEG data. In some embodiments, the application system can organize the training into epochs, each epoch including trials using all the EEG data in the training database. In some embodiments, the application system can organize epochs into batches including multiple trials. Consistent with disclosed embodiments, and as shown in FIG. 2A, the application system can perform a trial by applying an input EEG timeseries portion to the combined machine learning model to generate a predicted output EEG timeseries portion. The application system can determine a trial result by comparing the predicted output EEG timeseries portion to the actual corresponding portion of the input EEG timeseries. The application system can determine a loss value for a batch using a loss function and the results of the trials included in the batch. The application system can update the combined machine learning model using the loss value. The disclosed embodiments are not limited to a particular loss function. In some embodiments, the loss function can be a cross-entropy loss function, or another suitable loss function. Consistent with disclosed embodiments, the application system can perform training until a suitable duration, resource use, or performance criterion (e.g., cross-entropy loss value, or the like) is satisfied.

Consistent with disclosed embodiments, the application system can jointly train the tokenizing model, the sequential model, and the detokenizing model. The application system can determine updates to the tokenizing model, the sequential model, and the detokenizing model using the loss value for a batch and a suitable back-propagation method. In some embodiments, the application system can then apply the determined updates to the tokenizing model, the sequential model, and the detokenizing model. In some embodiments, the sequential model can be frozen. For example, the application system may only apply the determined updates to the tokenizing and detokenizing models.

FIG. 2B depicts a classification method 201 suitable for training or inference using components of the exemplary combined machine learning model of FIG. 2A, in accordance with disclosed embodiments. Classification method 201 can generate a classification using a classifier, components of a combined machine learning model, and an input portion of EEG timeseries data. The classification can be used in training the classifier, or to support patient diagnosis or classification.

As described herein, the combined machine learning model can be trained to predict future EEG timeseries data based on current EEG timeseries data. As appreciated by the inventors, training the combined machine learning model to predict future EEG timeseries data trains the combined machine learning model to represent the latent structure of the EEG timeseries data in the configuration of the machine learning model. As the latent structure of the EEG timeseries data reflects the underlying physiological processes that generated the EEG timeseries data, the configuration of the combined machine learning model may also reflect those processes, similar to the manner in which an observer can simulate the plant in a state-space controller. Classification method 201 can exploit this latent structure in the combined machine learning model to classify patients based on EEG timeseries data acquired from the patients.

For convenience of explanation, classification method 201 is described as being performed by an application system (e.g., application system 120, or the like) using training data acquired from a patient data acquisition device (e.g., data acquisition device 110, or the like). Furthermore, method 201 is depicted as being performed on EEG data. However, the disclosed embodiments are not so limited. For example, method 201 could be performed entirely on a patient data acquisition device. As an additional example, method 201 could be performed using a combined machine learning model trained to predict any suitable timeseries data (e.g., other electrical biosignals, speech data, or the like) or sequential data (e.g., 2D slices of 3D image data, or the like).

Furthermore, while FIG. 2B depicts classification method 201 using components of the combined model depicted in FIG. 2A (e.g., EEG tokenizer 210 and sequential model 230), the disclosed embodiments are not limited to such an architecture or to a combined model trained according to method 200. For example, method 201 can be used with a combined model trained by another suitable method or having an architecture differing from the architecture depicted in FIG. 2B.

Similar to method 200, method 201 can include an operation of obtaining input EEG timeseries data. Consistent with disclosed embodiments, the input EEG timeseries data can be directly or indirectly obtained from a patient data acquisition device. The patient data acquisition device can obtain the input EEG timeseries data, or signals used to generate the input EEG timeseries data, from patient sensors.

Consistent with disclosed embodiments, the application system can generate output features using the sequential model and the input token. As described herein, in various embodiments the output features can depend on values of components of the sequential model, or can be the output token(s) generated by the sequential model.

In an operation of method 201, the application system can generate a classification (e.g., classification 299B) by applying the output features to a classifier (e.g., classifier 240). The classifier can be a statistical or machine learning model, such as a support vector machine, linear or logistic regression classifier, decision tree or random forest model, linear or non-linear neural network, ensemble model including one or of any of the prior models, or another suitable statistical or machine learning model. In some embodiments, the output features can include internal state information of the sequential machine learning model or a function of the internal state information of the sequential machine learning model. For example, the output features can be or depend upon activation values for one or more layers of the sequential machine learning model. In some embodiments, the output features can include output token(s) generated by the sequential machine learning model.

In some embodiments, the classifier can be a sequential classifier. For example, the classifier can be configured to accept as input a sequence of output features. For example, such a sequential classifier can be configured to generate a new output token using a new set of output features and a prior model state or set of output features. In some embodiments, the sequential classifier can be implemented using an attention-based model (e.g., a transformer-based model, or the like), a state-space model, or a recurrent neural network.

In some embodiments, the classification can be an indication of a neurological state, disease, dysfunction, or injury of the subject. The neurological state, disease, dysfunction, or injury can be a brain age or a status, classification, or prognosis for epilepsy, concussion, stroke, traumatic brain injury, dementia, or Parkinsons. For example, the neurological state, disease, dysfunction, or injury can be a status of the subject as having Parkinsons. As an additional example, the neurological state, disease, dysfunction, or injury can be a classification of the subject as having a particular concussion type. As an additional example, the neurological state, disease, dysfunction, or injury can be a traumatic brain injury or stroke prognosis.

The disclosed embodiments are not limited to any particular form of the indication of neurological state, disease, dysfunction, or injury. In some embodiments, the classifier can be configured as a binary classifier and the provided indication can be a binary value. In some embodiments, the classifier can be configured as a multi-class classifier and the provided indication can specify one or more of three or more predetermined classes. In some embodiments, the classifier can be a sequence-to-sequence model and the provided indication can be a textual output.

In some embodiments, method 201 can be used in performing supervised training of the classifier. The application system can train the classifier using an obtained database of labeled EEG data. EEG data acquired from a subject can be labeled with an indication of a neurological state, disease, dysfunction, or injury of the subject. The application system can perform the training until a suitable duration, resource use, or performance criterion (e.g., cross entropy loss value, or the like) is satisfied.

As may be appreciated, the form of the supervised training can depend on the implementation of the classifier. For example, if the classifier is a neural network, the training can proceed over epochs that include batches of trials, as described above with regards to the training of the combined machine learning model. The application system can determine a trial result by comparing the classifier output (e.g., generated according to method 201) with the label for the EEG data used to generate the classifier output. The application system can determine a loss value for a batch using a loss function and the results of the trials included in the batch. The application system can update the classifier using the loss value. The disclosed embodiments are not limited to a particular loss function (which may differ from the loss function used to train the combined machine learning model). As may be appreciated, training may proceed differently for other types of models described herein (e.g., random forest models, support vector machine, linear or logistic regression classifier, decision tree or random forest models, etc.).

In some embodiments, method 201 can be used with a trained classifier to aid in the diagnosis or identification of patient neurological states, diseases, dysfunctions, or injuries. The application system can generate an indication of a neurological state, disease, dysfunction, or injury according to method 201 using EEG data acquired from the patient, the tokenizing model, sequential model, and the trained classifier. The application system can provide the indication to a user (e.g., the patient, a clinician, a caregiver or family member of the patient, or the like), or to a user device (which can in turn provide the indication to the user). The indication can be provided to the user through a graphical user interface as a textual or graphical indication. Additionally or alternatively, the indication can be provided to the user through another modality, such as an auditory or tactile modality. In some embodiments, the indication can be provided to a medical records system (e.g., medical records system 130). The medical records system may then update a medical record of the patient based on the received indication. For example, in response to an indication that a patient has Parkinsons, the medical records system can update a medical record of the patient to indicate that the patient has Parkinsons, or that the patient has been diagnosed with Parkinsons, or that a subject measuring system has classified the patient has having Parkinsons, or the like.

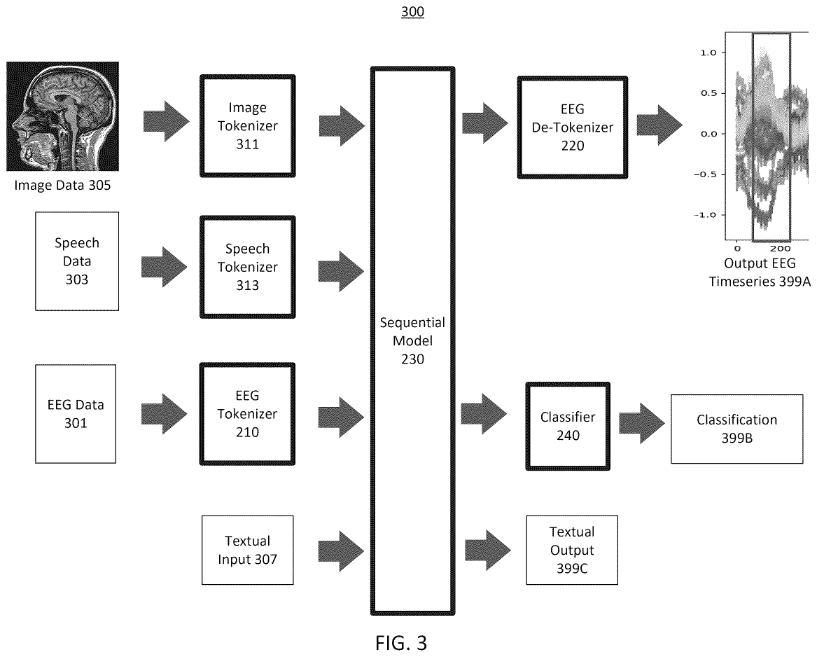

FIG. 3 depicts inputs, outputs, and components of an exemplary multimodal combined machine learning model 300 suitable for training and inference, in accordance with disclosed embodiments. Similar to the combined machine learning model depicted in FIG. 2A, combined machine learning model 300 can include a sequential model pre-trained to perform language processing tasks (e.g., sequential model 230, or the like). In some embodiments, combined machine learning model 300 can include encoder models (e.g., image tokenizer, speech tokenizer, EEG tokenizer, or the like) that encode input data samples in various input domains (e.g., images, EEG timeseries data, speech timeseries data, or the like) into tokens in an internal vocabulary of the sequential model. The combined machine learning model 300 can further include de-tokenizing models or classifiers that convert tokens in the internal vocabulary of the sequential model to outputs in suitable output domains (e.g., EEG data, Speech data, class indicators, or the like).

While machine learning model 300 is depicted as being configured to process EEG timeseries data, the disclosed embodiments are not so limited. Additionally or alternatively, the combined machine learning model 300 can be configured to process other timeseries data, such as speech timeseries data.

In some embodiments, combined machine learning model 300 can be configured to accept EEG timeseries inputs. In such embodiments, combined machine learning model 300 can further include an EEG tokenizing model (e.g., EEG tokenizing model 210, or the like) and a de-tokenizing model (e.g., EEG detokenizing model 220, or the like) configured to adapt the sequential model to EEG processing tasks. Combined machine learning model 300 can be configured to obtain an input EEG timeseries (e.g., EEG 301) similar to input EEG timeseries 202A and input EEG timeseries 202B.

In some embodiments, the multimodal combined machine-learning model 300 can include non-EEG input modalities. Such modalities can include textual inputs (e.g., textual input 307), auditory inputs (e.g., speech input 303), image inputs (e.g., image data 305), or other suitable input modalities. The multimodal combined machine-learning model 300 can use these inputs to configure the sequential model or as data sources, thereby improving the diagnosis or identification of patient neurological states, diseases, dysfunctions, or injuries.

In some embodiments, machine-learning model 300 can be configured to process textual input 307. In some embodiments, textual input 307 can include instructions (e.g., prompts, or the like). The instructions can specify a context for the sequential model. For example, such instructions could state: “You are a friendly and helpful biomedical assistant who can read and interpret EEG data and use it to answer questions.” As an additional example, such instructions could state: “I have provided 1 second of EEG from a participant. Please predict the next second of their EEG and describe their health and state of mind.” The instructions can provide information about other input data. For example, the instructions can specify the modalities of input data sources (e.g., EEG or speech) or tokens (e.g., “the input includes a sequence of ten EEG tokens followed by ten speech tokens” or the like). The instructions can limit the sequential model to considering particular temporal or signal-source portions of the input data. For example, instructions can limit the sequential model to considering only the first second of EEG timeseries input data, or only particular channels of EEG timeseries input data (e.g., “Please predict the next second of patient EEG using only the T3 and T4 electrode channels.”). In some embodiments, when the machine learning model 300 includes a textual output, the instructions can specify the contents of the textual output. For example, the instructions can specify a format, schema, tool call definition, or the like for the textual output.

In some embodiments, textual input 307 can include medical data for a subject. In some embodiments, the application system can obtain textual input 307 stored in configuration files, program code, databases on the application system or other computing systems (e.g., medical records stored on medical records system 130) or the like. In some embodiments, the application system can obtain textual input 307 from a user through interactions with a user interface of the application system or a user device (e.g., user device 140). In some embodiments, the application system can combine textual data from multiple sources (e.g., medical records, configuration files, program code, user interactions, or the like) into textual input 307.

In some embodiments, machine-learning model 300 can be configured to process speech data 303. In some embodiments, speech data 303 can be time domain data in a suitable format (e.g., raw audio data in formats such as WAV, AIFF, or the like; decoded versions of lossy or lossless compressed audio data, such as FLAC, MP3, MPEG-4 ALS, or the like). In some embodiments, speech data 303 can be frequency domain data (e.g., Mel-frequency Cepstral Coefficients, spectrogram data, or other suitable frequency domain representations of speech). The application system can directly or indirectly obtain the speech data from a microphone configured to acquire the speech of the patient. For example, the microphone can be part of a user device (e.g. user device 140, or the like), a data acquisition device (e.g., data acquisition device 110, or the like), or another device (e.g., a recording device used by a clinician to acquire the speech data, or the like).

In some embodiments, machine-learning model 300 can be configured to process image data 305. The disclosed embodiments are not limited to any particular digital format or type of image data. In some embodiments, the image data can be JPEG, JPEG 2000, TIFF, or another suitable format. In some embodiments, the image data can be radiographic images, magnetic resonance imaging images, scintigraphic images, single-photon emission computed tomography images, positron emission tomography images, ultrasound images, or other suitable image types. In some embodiments, the images can be images of the brain or central nervous system of the patient.

In an operation, the application system can obtain multimodal input data. In some embodiments, this input data can include two or more of EEG data 301, speech data 303, and image data 305. In some embodiments, this input data can further include textual data. In some embodiments, the application system can obtain the input data at the same time. For example, the application system can obtain a database including training samples. The training samples can be multimodal sets of labeled training data. In some embodiments, the application system can obtain the input data at different times. For example, textual data providing instructions can be obtained prior to other input data (e.g., the application system can obtain instructions stored in a configuration file or program code). As an additional example, EEG data or speech data can be continuously acquired from a subject, while image data or textual data can be occasionally obtained. For example, the application system can obtain image data or textual data from a medical record of the subject. The application system can retrieve or receive such data from medical records system 130, or the like. The application system can retrieve or receive such data in response to a command received by the application system, based on user input provided to a user device, automatically when a condition is satisfied, or for another suitable reason.

In an operation, the application system can provide the input data to corresponding components of combined machine learning model 300. In some embodiments, when the input data includes textual input 307, the application system can provide this textual input to sequential model 230.

In some embodiments, when the input data includes EEG Data 301, the application system can provide this data to EEG tokenizer 210, which can generate tokens as described herein with regards to FIGS. 2A and 2B.

In some embodiments, when the input data includes speech data 303, the application system can provide speech data 303 to speech tokenizer 313. Similar to EEG tokenizer 210, speech tokenizer 313 can be a machine learning model, such as a feed-forward neural network. Similar to EEG tokenizer 210, speech tokenizer 313 can be configured to accept as input a first number of speech data 303 samples and output a lesser number of tokens. In some embodiments, speech tokenizer 313 can be configured to accept 2-64 samples, or more, sampled with a sampling period of 1 to 50 ms (or in the case of frequency data, corresponding to 1 to 50 ms of speech data) into a single token.

Similar to EEG tokenizer 210, speech tokenizer 313 can be trained and configured to adapt combined machine learning model 300 to the performance of speech processing tasks. The input of speech tokenizer 313 can be suitable for speech processing tasks. For example, the input of the tokenizing model can be scalars or vectors of amplitude values (e.g., time-domain signal measurements, frequency domain measurements for multiple frequency values, or the like.

In some embodiments, the output of speech tokenizer 313 can be adapted to a language processing task, similar to EEG tokenizer 210. For example, the tokenizing model can be configured to output a token included within the internal vocabulary of sequential model 230. In some embodiments, the speech tokenizer 313 can be configured to output a token in vocabulary differing from the internal vocabulary of sequential model 230. The application system can then convert this token into a token in the internal vocabulary of the sequential model.

In some embodiments, when the input data includes image data 305, the application system can provide image data 305 to image tokenizer 311. Similar to EEG tokenizer 210, image tokenizer 311 can be a machine learning model. In some embodiments, image tokenizer 311 can include a feed-forward convolutional neural network. Image tokenizer 311 can be configured to accept an image (which may include multiple channels, such as red, green, and blue channels for an RGB image) and output one or more tokens.

Similar to EEG tokenizer 210, image tokenizer 311 can be trained and configured to adapt combined machine learning model 300 to the performance of image processing tasks. The input of image tokenizer 311can be suitable for image processing tasks. For example, the input of image tokenizer 311 can be one or more channels of image data (e.g., three matrices of pixel amplitude values, a first matrix corresponding to red pixel amplitude values, a second matrix corresponding to green pixel amplitude values, a third matrix corresponding to blue pixel amplitude values).

In some embodiments, the output of speech tokenizer 313 can be adapted to a language processing task, similar to EEG tokenizer 210. For example, image tokenizer 311 can be configured to output a token included within the internal vocabulary of sequential model 230. In some embodiments, image tokenizer 311 can be configured to output a token in vocabulary differing from the internal vocabulary of sequential model 230. The application system can then convert this token into a token in the internal vocabulary of the sequential model.

In an operation, the application system can apply a sequence of input tokens to sequential model 230. This sequence of input tokens can include input tokens from multiple different input sources. For example, as described herein, such input tokens can be included in textual input 307, or generated using an input-data-appropriate tokenizing model (e.g., image tokenizer 311 for image data, speech tokenizer 313 for speech data, or EEG tokenizer 210 for EEG data).

In some embodiments, the application system can provide tokens to sequential model 230 as the tokens are obtained. For example, when the EEG tokenizer and the speech tokenizers are concurrently generating tokens, the application system can combine these tokens as they are generated into a single input sequence applied to sequential model 230.

In some embodiments, the application system can group tokens from certain sources (e.g., EEG, speech, textual instructions, or the like) together into subsequences. For example, when the EEG tokenizer and the speech tokenizers are concurrently generating tokens, the application system can accumulate these tokens into subsequences (e.g., having predetermined token lengths) and combine these subsequences into the single input sequence applied to sequential model 230.

In some embodiments, the sequence of input tokens can include information indicating an input token modality. For example, the sequence of input tokens can include delimiters indicating whether tokens were obtained from textual input 307 or obtained from a particular tokenizing model. In some embodiments, the application system can be configured to insert such tokens into the sequence of input tokens. For example, given tokens [t1] and [t2] obtained from EEG tokenizer 210, tokens [t3] and [t4] obtained from speech tokenizer 313, token [t5] obtained from image tokenizer 311, and textual input tokens [and], [use], [it], [to], [answer], [question], [s], the application system can generate an input sequence S:
S=[_text], [and], [use], [it], [to], [answer], [question], [s], [_image], [t5], [_speech], [t3], [t4], [_EEG], [t1], [t2]
   As may be appreciated, the application system can insert the token modality delimiters [_text] for textual input, [_image] for image tokens, [_speech] for speech tokens, and [_EEG] for EEG tokens can be inserted into the input sequence of tokens provided to sequential model 230. While this example depicts token modality delimiters corresponding to all input types, the disclosed embodiments are not so limited. In some embodiments, only certain input modalities can be associated with token modality delimiters. In some embodiments, the tokenizing models can be trained and configured to output token modality delimiters indicating the source of the tokens that they generate.

Consistent with disclosed embodiments, the application system can generate output tokens from the sequence of input tokens using sequential model 230. As described with regards to FIGS. 2A and 2B, sequential model 230 can be a sequential neural network model. In some embodiments, sequential model 230 can be an attention-based model (e.g., a transformer-based model, or the like), a state-space model, or a recurrent neural network. In some embodiments, the sequential neural network model can be configured to combine one or more new input tokens with an internal state to generate an updated internal state or output token. In some embodiments, the sequential neural network model can be configured to sequentially add new input tokens to a context window, or slide a context window along a sequence of input tokens. As described herein, sequential model 230 can be a pretrained language model. The disclosed embodiments are not limited to any particular architecture. In some embodiments, sequential model 230 can be an implementation of the MAMBA, LLAMA, or GWEN architectures. As described with regards to FIGS. 2A and 2B, sequential model 230 can generate output features when generating the output tokens. In some embodiments, as described herein, these output features can be based on the values of components of sequential model 230.

In an operation, the application system can generate multimodal outputs. For example, the application system can generate textual outputs (e.g., textual output 399C), EEG outputs (e.g., output EEG timeseries 399A), speech outputs, or classifications (e.g., classification 399B). In some embodiments, the application system can apply certain tokens to de-tokenizing models to generate the EEG or speech outputs. In some embodiments, the application system can apply output features (which may include certain tokens) to a classifier (e.g., classifier 240, or the like) to generate classifications (e.g., classification 399B).

In some embodiments, the application system can determine output tokens provided as textual outputs, applied to de-tokenizing models, or applied to classifiers. In some embodiments, the application system can perform this determination using modality delimiters included in the sequence of output tokens. For example, sequential model 230 can generate a sequence of output tokens:
S=[_EEG], [o1], [o2], [_speech], [o3], [o4], [_text], [MMSE], [Score], [:]. [19]
   where tokens [o1] and [o2] encode EEG information, tokens [o3] and [o4] encode speech information, and tokens [MMSE], [Score], [:]. [19] are textual output tokens. The application system can be configured to parse the sequence of output tokens, routing output tokens to appropriate destinations. For example, the application system can convert the textual output tokens into text (e.g., by combining tokens into words and adding whitespace, or another suitable method). As an additional example, the application system can input tokens encoding EEG information to EEG De-Tokenizer 220. As an additional example, the application system can input tokens encoding speech information to a speech de-tokenizing model (not shown in FIG. 3).

In some embodiments, when the application system generates output EEG timeseries 399A, the relationship between this data and EEG Data 301 can be similar to the relationship between output EEG timeseries 299A and input EEG timeseries 202A. For example, output EEG timeseries 399A can be a portion of EEG timeseries data predicted based on the input EEG Data 301. As described herein with regards to FIGS. 2A and 2B, training combined machine learning model 300 to predict EEG data can cause combined machine learning model 300 to develop an internal representation of latent structures of EEG timeseries data. The application system can use such latent structures to identify a neurological state, disease, dysfunction, or injury of a subject based at least in part on EEG data obtained from that subject.

In some embodiments, when the application system generates output speech data, the relationship between this data and speech data 303 can be similar to the relationship between output EEG timeseries 299A and input EEG timeseries 202A. For example, the output speech data can be a portion of speech data predicted based on the speech data 303. As with EEG data, training combined machine learning model 300 to predict speech data can cause combined machine learning model 300 to develop an internal representation of latent structures of speech data. The application system can use such latent structures to identify a neurological state, disease, dysfunction, or injury of a subject based at least in part on speech data obtained from that subject.

In some embodiments, similar to classification 299B, classification 399B can be an indication of a neurological state, disease, dysfunction, or injury of the subject. As described herein, the neurological state, disease, dysfunction, or injury can be a brain age or a status, classification, or prognosis for epilepsy, concussion, stroke, traumatic brain injury, dementia, or Parkinsons. The disclosed embodiments are not limited to any particular form of the indication of neurological state, disease, dysfunction, or injury.

In some embodiments, textual output 399C can be text generated using tokens output by sequential model 230. Combined machine learning model 300 can be trained to generate textual output 399C relevant to the determination of a neurological state, disease, dysfunction, or injury of a subject. For example, combined machine learning model 300 can be trained to output a predicted neurological assessment value (e.g., “MMSE score: 18”) or a predicted status of the patient (e.g., “Brain age: 77”).

In some embodiments, the application system can be configured to combine outputs into a predetermined output format. For example, the application system can be configured to combine a textual introduction (which may be preconfigured or generated using combined machine learning model 300) with one or more of textual output 399C, classification 399B, or speech output, or output EEG timeseries 399A. The application can be configured to format output data for presentation (e.g., displaying speech output or output EEG timeseries 399A as a plot with appropriate axis, divisions, legends, or the like).

In an operation, the application system can display, store, or use the outputs of combined machine learning model 300, or provide such outputs to another computing system for display, storage, or use. For example, the application system can provide such outputs to a user device (e.g., user device 140) for display in a graphical user interface to a user. As an additional example, when the application system obtains input data for a subject (e.g., EEG data, image data, speech data), the application system can provide outputs generated using this input data to a medical records system (e.g., medical records system 130) for use in updating a medical record of the subject. As a further example, the application system can use the outputs as prompts for automatically performing additional tests or as triggers for automated alerts.

In some embodiments, training of the combined machine learning model 300 can be performed using the input data and components depicted in FIG. 3. The training can include supervised or unsupervised training.

In some embodiments, the training can include an initial unsupervised training step, followed by a supervised training step. In the initial unsupervised training step, the application system can jointly train components of combined machine learning model 300 (e.g., tokenizing model(s), sequential model 230, detokenizing model(s), or the like) to predict timeseries output data from timeseries input data. In some embodiments, the application system can jointly train these components using a multimodal training dataset. In some embodiments, the jointly trained components can include a tokenizing model and a detokenizing model for each timeseries input modality (e.g., EEG data, speech data, or the like). As described herein with regards to FIG. 2A, the application system can generate output data predictions in a trial using input data and the jointly trained components of combined machine learning model 300. The application system can determine trial results by comparing the output data predictions and the corresponding portions of the input timeseries data. The application system can update the components based on the trial results for a batch of trials. For example, the application system can determine a loss value based on the trial results. The application system can then determine updates to the jointly trained components based on the loss value. In some embodiments, the sequential model 230 can be frozen during training, as described herein. In some embodiments, sequential model 230 can be updated during training. In some embodiments, the application system can perform multiple epochs of training. Consistent with disclosed embodiments, the application system can perform training until a suitable duration, resource use, or performance criterion is satisfied.

In some embodiments, in the supervised training step, the application system can jointly train components of combined machine learning model 300 (e.g., sequential model 230, classifier 240, or the like) using a multimodal training dataset including labeled training examples. The labeled training examples can include EEG or speech timeseries input data (e.g., EEG Data 301 or Speech Data 303). In some embodiments, the labeled training examples can include image or textual input data. Consistent with disclosed embodiments, the labels can include ground-truth classifications or textual outputs. The application system can generate predicted classifications or textual outputs using the jointly trained components and the timeseries input data (and, in some embodiments, the image or textual input data). The application system can determine trial results by comparing the predicted classifications or textual outputs with the ground-truth classifications or textual outputs. In some embodiments, the multimodal training dataset can include textual input data. In some embodiments, as described herein, this textual input data can include instructions specifying the format or content of the output of combined machine learning model 300. For example, such instructions can specify a format, schema, tool call definition, or the like for the textual output. As may be appreciated, the ground-truth classifications or textual outputs for a labeled training example can reflect the instructions in the corresponding textual input data. The application system can update the components based on the trial results for a batch of trials. For example, the application system can determine a loss value based on the trial results. The disclosed embodiments are not limited to a particular loss function. In some embodiments, the loss function can be a cross-entropy loss function, or another suitable loss function. In some embodiments, when the application system generates multiple outputs, individual loss values can be generated for each output. An overall loss value can be generated as a function (e.g., a weighted average) of these individual loss values. The application system can then determine updates to the jointly trained components based on the overall loss value. In some embodiments, the sequential model 230 can be frozen during training, as described herein. In some embodiments, sequential model 230 can be updated during training. In some embodiments, the application system can perform multiple epochs of training. Consistent with disclosed embodiments, the application system can perform training until a suitable duration, resource use, or performance criterion is satisfied.

In some embodiments, the application system can combine supervised and unsupervised training. For example, the application system can obtain a multimodal training dataset including labeled training examples. The labeled training examples can include timeseries input data. In some embodiments, the labeled training examples can include image input data or textual input data. The application system can jointly train tokenizing models, de-tokening models, the sequential model, and classifiers using the labeled training examples. For example, the application system can determine trial results by comparing predicted timeseries output data to corresponding portions of input timeseries data and predicted textual output or classifications can be compared to ground-truth textual output or classifications. The application system can update the components based on the trial results for a batch of trials. For example, the application system can determine a loss value based on the trial results. The disclosed embodiments are not limited to a particular loss function. In some embodiments, the loss function can be a cross-entropy loss function, or another suitable loss function. In some embodiments, when the application system generates multiple outputs, individual loss values can be generated for each output. An overall loss value can be generated as a function (e.g., a weighted average) of these individual loss values. The application system can then determine updates to the jointly trained tokenizing models, de-tokening models, the sequential model, and classifiers based on the overall loss value. Consistent with disclosed embodiments, the application system can perform training until a suitable duration, resource use, or performance criterion is satisfied.

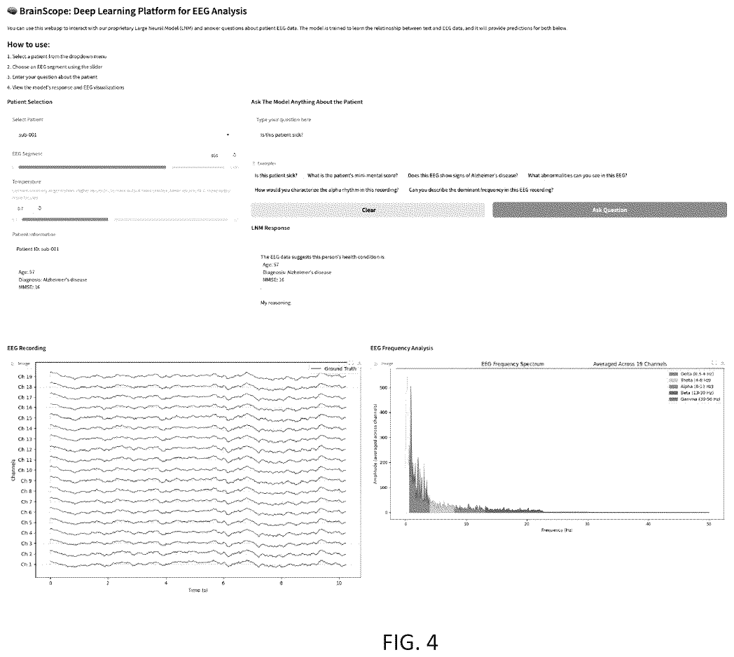

FIG. 4 depicts a graphical user interface for a subject measuring system, in accordance with disclosed embodiments. Such a graphical user interface may be displayed by a component of the subject measuring system (e.g., data acquisition system 110, application system 120, user device 140, or the like of subject measuring system 100). As depicted in FIG. 4, the graphical user interface can enable a user to select a subject and select an EEG segment for the subject. In this example, “patent information” displays information for the subject (e.g., age, diagnosis, MMSE score). As may be appreciated, some or all of this information may be absent in other embodiments. The graphical user interface enables the user to enter textual input. In this example, the textual input can be questions, though instructions and data can also be provided, consistent with disclosed embodiments. As may be appreciated, the provided textual input can be applied to the machine learning model (e.g., together with the EEG segment) to generate a machine learning model output. In this example, the output correctly states a predicted age, diagnosis, and MMSE score for this subject.

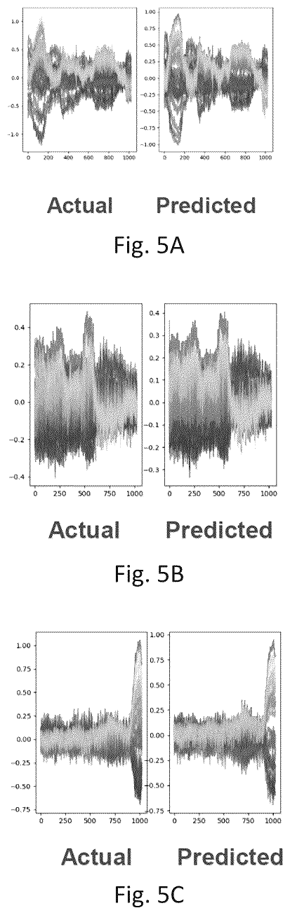

FIGS. 5A to 5C depict exemplary comparisons between actual EEG outputs and EEG outputs generated using a combined machine learning model, in accordance with disclosed embodiments. As can be observed from these examples, such a machine learning model can predict EEG timeseries data based on input EEG timeseries data.

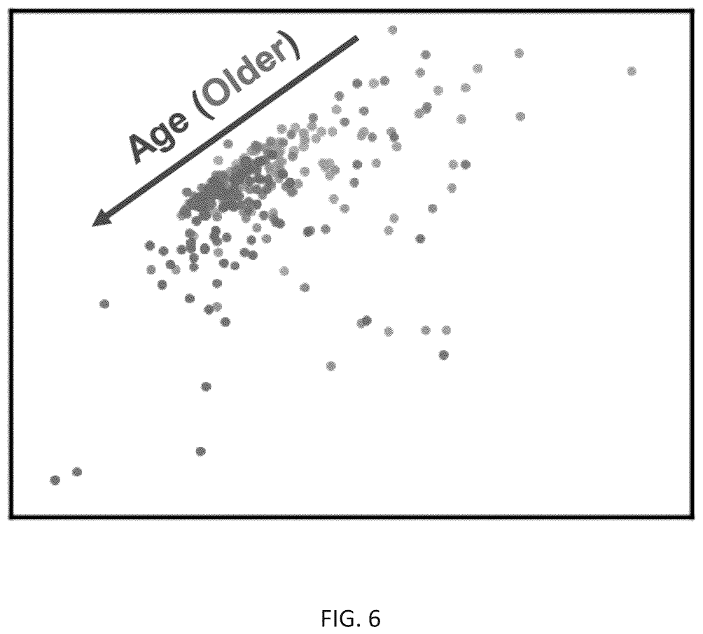

As depicted in FIG. 6, a combined model trained consistent with disclosed embodiments can generate output tokens encoding subject age information. In this example, a combined machine learning model similar to the model depicted in FIG. 2A generated average subject values for multiple subjects using subject EEG timeseries data. The combined machine learning model included a sequential model that generated multiple output tokens for each subject using the input EEG data for the subject. These output tokens were averaged to generate a subject vector. The subject vectors were concatenated to form a subject matrix. The subject matrix was mapped to a two-dimensional space using a suitable dimensionality reduction procedure. FIG. 6 depicts this mapping, with data points representing subjects having label types dependent on ground-truth subject age. As apparent from FIG. 6, subject age increases towards the lower left of this mapping, showing that the output tokens generated by the sequential model encoded information about the age of the subjects.

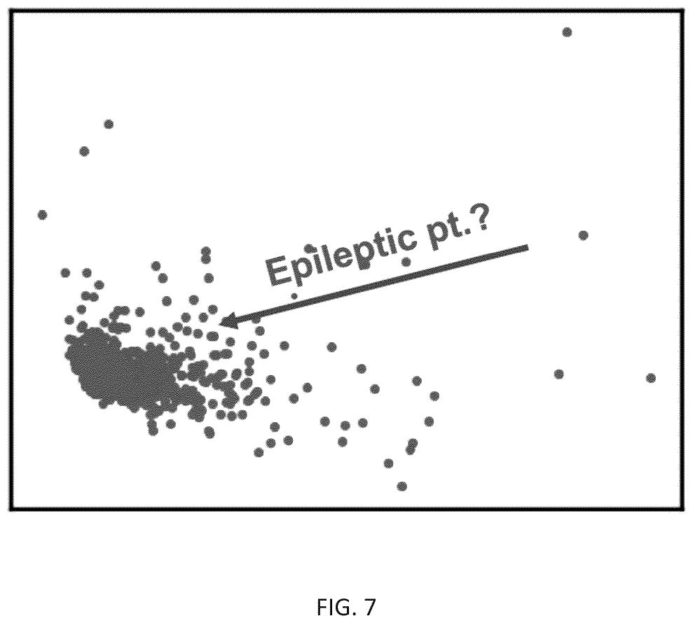

As depicted in FIG. 7, a combined model trained consistent with disclosed embodiments can generate output tokens encoding an epileptic status of a subject. As described with regards to FIG. 6, a combined machine learning model similar to the model depicted in FIG. 2A generated average subject values for multiple subjects using subject EEG timeseries data. As described with regards to FIG. 6, these average subject values were mapped to a two-dimensional space using a suitable dimensionality reduction procedure. FIG. 7 depicts this mapping, with data points representing subjects having label types dependent on ground-truth epileptic status. As apparent from FIG. 7, epileptic patients are clustered in a particular region of the two-dimensional space, showing that the output tokens generated by the sequential model encoded information about the epileptic status of the subjects.

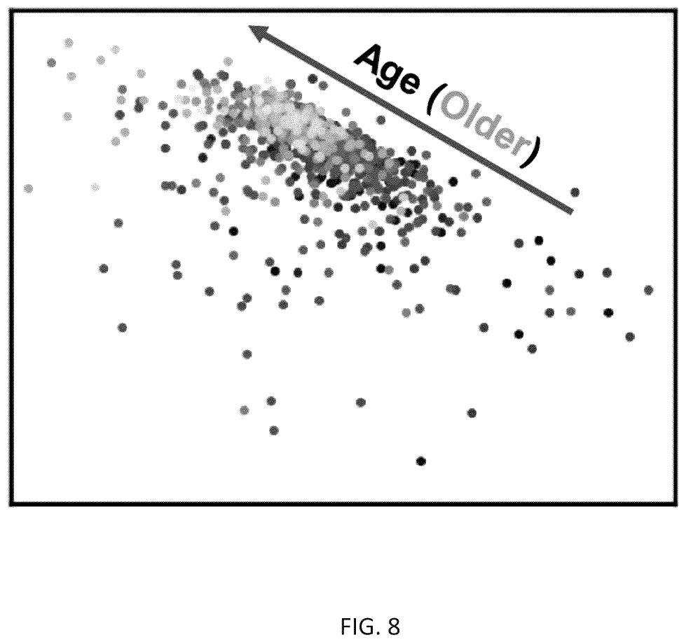

As depicted in FIG. 8, a combined model trained consistent with disclosed embodiments may use patient age among other factors in assessing epileptic status. As described with regards to FIG. 6, a combined machine learning model similar to the model depicted in FIG. 2A generated average subject values for multiple subjects using subject EEG timeseries data. The average subject values were mapped to a two-dimensional space using a partial least squares regression with epileptic status and subject age as responses. FIG. 8 depicts this mapping, with data points representing subjects having label types dependent on ground-truth subject age. A correlation between ground truth subject age and epileptic status is apparent, showing that the combined model considers patient age in determining epileptic status.

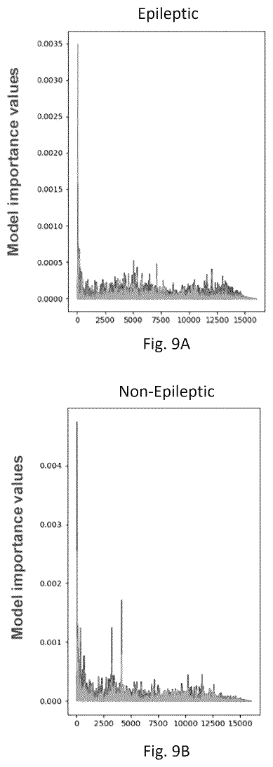

FIGS. 9A and 9B depict the importance to a combined model trained consistent with disclosed embodiments of portions of EEG timeseries data in determining average subject values for epileptic and non-epileptic patients. In both graphs, the x-axis is timeseries sample number and the y-axis is relative model importance. As described with regards to FIGS. 6 to 8, EEG timeseries data for a subject was used to generate an average subject value for the subject. The average subject value was used to estimate the importance each input sample for each EEG channel to the generated average subject value. As depicted in FIGS. 9A and 9B, initial samples were extremely important in determining the average subject value, but the model used samples throughout the duration of the trial in determining the output average subject value.

### Exemplary Device for Identifying Neurological State, Disease, Dysfunction, or Injury

Neurological state, disease, dysfunction, or injury can be identified, consistent with disclosed embodiments, using devices configured to acquiring EEG signals from patients. The disclosed systems and methods can be practiced using a field-portable EEG device designed and adapted for use in outpatient clinics, emergency rooms, or at other point-of-care settings. Such a device may be able to perform neurological state, disease, dysfunction, or injury identification as described herein without requiring the assistance of a skilled technician. Combined with the systems and methods of neurological state, disease, dysfunction, or injury identification described herein, such a device may provide an improved ability to perform diagnosis and assessment of subjects (e.g., subjects with suspected neurological disease, dysfunction, or injury).

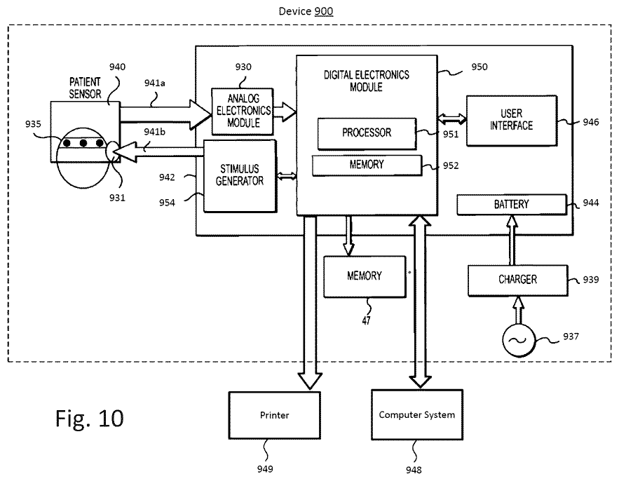

FIG. 10 depicts an exemplary schematic of a device 1000 for identifying neurological state, disease, dysfunction, or injury, consistent with disclosed embodiments. In some embodiments, device 1000 can be a component of a subject measuring system (e.g., subject measuring system 100). In such embodiments, device 1000 can be a data acquisition device (e.g., data acquisition device 110). Device 1000 can be configured to generate EEG timeseries data by acquiring and processing brain electrical signals. In some embodiments, device 1000 can provide this EEG timeseries data to another system configured to identify neurological state, disease, dysfunction, or injury (e.g., application system 120, user device 140, or the like). In some embodiments, device 1000 can be configured with a machine learning model trained, as described herein with regards to FIGS. 2A, 2B, and 3, to identify neurological state, disease, dysfunction, or injury. In some embodiments, device 1000 can obtain the machine learning model from another system (e.g., application system 120). In some embodiments, device 1000 can obtain medical data for identifying neurological state, disease, dysfunction, or injury from another system (e.g., medical records system 130, application system 120, or the like). In some embodiments, device 1000 can display the identification of neurological state, disease, dysfunction, or injury, or provide the identification to another component of subject measuring system 100 (e.g., application system 120, medical records system 130, user device 140, or the like). In an exemplary embodiment, the device 1000 can be implemented as a portable device for point-of-care applications. Device 1000 can include patient sensor 940, which may be coupled to a base unit 942, which can be handheld, as illustrated in FIG. 9. Patient sensor 940 may include an electrode array 935 comprising at least one disposable neurological electrode to be attached to a patient's head to acquire brain electrical signals. In some embodiments, the electrodes can be configured for sensing both spontaneous brain activity as well as evoked potentials generated in response to applied audio stimuli. In some embodiments, the device 1000 can include five (active) channels and three reference channels. The electrode array 1035 can include anterior (frontal) electrodes: F1, F2, F7, F8, AFz (also referred to as Fz′) and Fpz (reference electrode) to be attached to a subject's forehead, and electrodes A1 and A2 to be placed on the front or back side of the ear lobes, or on the mastoids, in accordance with the International 10/20 electrode placement system (with the exception of AFz).

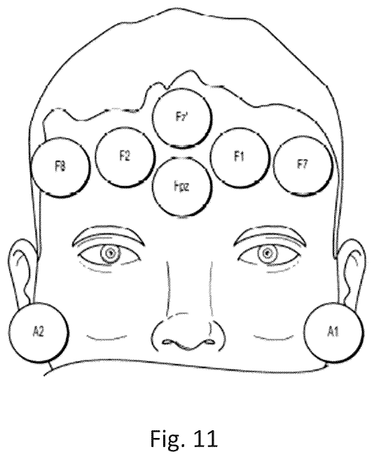

FIG. 11 depicts an exemplary placement of device 1000 on the head of a patient, consistent with disclosed embodiments. The use of a limited number of electrodes can enable rapid and repeatable placement of the electrodes on a subject, which in turn facilitates efficient, and more accurate, patient monitoring. Further, in some embodiments, the electrodes may be positioned on a low-cost, disposable platform, which can serve as a “one-size-fits-all” sensor. For example, electrodes 1035 may be positioned on a head gear that is configured for easy and/or rapid placement on a patient. The head gear may be single-use or disposable. As may be appreciated, the disclosed embodiments are not limited to embodiments using device 1000 or the electrode configuration displayed in FIG. 11.

Consistent with disclosed embodiments, patient sensor 1040 can include at least two reusable patient interface cables. These cables can be designed to plug into the base unit 1042 and provide direct communication between the patient sensor 1040 and the base unit 1042. The first cable can be an electrical signal cable 1041a, which can be equipped with standard snap connectors to attach to the disposable electrodes placed on the patient's scalp. The second cable can be cable 1041b, which can provide stimulii signals for evoking a response (e.g., mid-latency, late auditory responses, P300, or other suitable responses) from the patient (e.g., cable 1041b can provide a connection to the earphone 1031 for auditory stimulus delivery, or the like).

Consistent with disclosed embodiments, the base unit 1042 can include an analog electronics module 1030, a digital electronics module 1050, user interface 1046, stimulus generator 1054 and battery 1044 as illustrated in FIG. 10. In some embodiments, the analog electronics module can receive signals from one or more of the neurological electrodes operatively connected through the electrical cable 1041a. The analog module can be configured to amplify, filter, and preprocess the analog waveforms acquired from the electrode channels. The analog module may comprise signal amplifier channels including at least one preamplifier, at least one differential amplifier, at least one common mode detector, and at least one gain stage with filter. The analog amplifier channels correspond to the number of electrode channels. In some embodiments, there are 8 analog amplifier channels corresponding to 8 electrode channels (5 active, 3 reference channels). The analog module 1030 may further include a multiplexer (MUX), which combines many analog input signals and outputs them into a single channel, and an analog-to-digital converter (ADC) to digitize the received analog signal. Digital electronics module 1050 can then process the digitized data acquired through analog module 1030 and can perform analysis of data to aid in interpretation of data pertaining to brain electrical activity. Further, as shown in FIG. 10, the digital electronics module 1050 can be operatively connected with a number of additional device components.

In some embodiments, the analog electronics module 1030 can be further configured to check an impedance by feeding a signal back into each electrode. Checking an impedance may function to characterize the effectiveness of connection of a surface electrode to a subject. Such checking can enable a user to test the applied electrodes at a patient site before signal acquisition is started and also monitor the electrode impedance continuously in real-time throughout the test. In an exemplary embodiment, the impedance of the applied electrodes can be measured periodically, and the impedance values can be displayed on the user interface 1046 using a color-coded electrode visual display, which allows the user to monitor the electrode impedances before and during a test session. If an impedance value is found to be unacceptable at the time of the measurement, a warning indication may be displayed on the screen instructing the user to check the electrode connection.

The digital electronics module 1050 comprises a digital signal processor (DSP) 1051 for processing the data corresponding to the acquired brain electrical signals, and a memory 1052 which stores the instructions for processing the data, such as a DSP algorithm. In some embodiments, device 1000 can be configured to use module 1050 in performing a method as depicted in FIGS. 2A, 2B, and 3, or another similar method. For example, when configured with a suitable machine-learning model (e.g., stored in memory 1052, or another suitable location), digital electronics module 1050 can use DSP 1051 in:
a. Extraction and preprocessing of timeseries EEG data used by the machine-learning model; and
b. Identification of a neurological state, disease, dysfunction, or injury for the subject, as described herein.

Consistent with disclosed embodiments, DSP 1051 can be configured to discard data that is contaminated by non-brain-generated artifacts, such as eye movements, electromyographic activity (EMG) produced by muscle tension, spike (impulse), external noise, etc., as well as unusual electrical activity of the brain not part of the estimation of stationary background state. In some non-limiting embodiments, artifact identification is performed using as input the signals from the five active leads Fp1, Fp2, F7, F8, AFz referenced to linked ears (A1+A2)/2, and sampled at 100 Hz. As may be appreciated, the disclosed embodiments are not limited to particular methods of artifact removal or suppression.

In some embodiments, device 1000 (e.g., using digital electronics module 1052, or the like) can be configured to identify a neurological state, disease, dysfunction, or injury, as described herein. In some embodiments, device 1000 can be configured to display or provide (e.g., to user device 140) textual output (e.g., textual output 399C) or classifications (e.g., classification 399B).

Consistent with disclosed embodiments, memory 1052 can be configured to contain interactive instructions for using and operating device 1000. Device 1000 can be configured to (e.g., in response to suitable commands, or the like) display these instructions on a screen of the user interface 1046. The instructions may comprise an interactive feature-rich presentation including a multimedia recording providing audio/video instructions for operating the device, or alternatively simple text, displayed on the screen, illustrating step-by-step instructions for operating and using the device. The inclusion of interactive instructions with the device eliminates the need for extensive training for use, allowing for deployment and use by persons other than medical professionals. Memory 1052 may also contain a database, which can include obtained timeseries and medical data used in identifying a neurological state, disease, dysfunction, or injury. In an exemplary embodiment, the database may be accessed from a remote storage device via a wireless or a wired connection. Similarly, data collected from the patient by device 1000 may be recorded in the database for future reference.

Device 1000 can be a standalone system or can operate in conjunction with a mobile (e.g., user device 140) or stationary device (e.g., application system 120) to facilitate display or storage of data, and to signal healthcare personnel. Mobile devices can include, but are not limited to, handheld devices and wireless devices distant from, and in communication with, device 1000. Stationary devices can include, but are not limited to, desktop computers, printers and other peripherals that display or store the results of the neurological evaluation. In an exemplary embodiment, device 1000 can be configured to provide patient files (e.g., acquired timeseries data, or session and test results) to a computer 1048, which can be another component of the subject measuring system (e.g., application system 120, medical records system 130, user device 140, or the like) using a wired or wireless connection to a network (e.g., network 150). In some embodiments, the results can be transmitted wirelessly or via a cable to a printer 1049 that prints the results to be used by attending medical personnel. In some embodiments, user interface 1046 may be configured to communicate patient information and/or procedural data to medical personnel. Information that is conveyed through user interface 1046 can include a variety of different data types, including, but not limited to, diagnostic results, intermediate analysis results, usage settings, etc. In some embodiments, the user interface 1046 can display the brain electrical signal graphs drawn in real-time for the five active and the three reference channels.

In some embodiments, user interface 1046 can be configured to receive and display usage setting information, such as the name, age and/or other statistics pertaining to the subject. The user interface 1046 can include a touchscreen interface for entering the user input. A virtual keypad may be provided on the touchscreen interface for input of patient record fields. Additionally, user interface 1046 can be configured to display the battery charge status, available memory status of non-transitory computer-readable medium 1047, and electrode impedance values. Further, the neuro-assessment device can transmit data to another mobile or stationary device to facilitate more complex data processing or analysis. For example, the neuro-assessment device, operating in conjunction with computer 1048, can send data to be further processed by computer 1048. In some embodiments, device 1000 can transmit the raw, unprocessed signal acquired from a subject to computer 1048 for analyzing the recorded data and outputting the results. The unprocessed brain electrical signals recorded from a subject may also be stored in a remote database for future reference and/or additional signal processing.

In some embodiments, base unit 1042 can include a stimulus generator 1054, which is operatively coupled to DSP 1051, for applying auditory stimuli to the subject to elicit ABR waveforms. The stimulus generator 1054 can interface with earphone 1031 positioned in proximity to the patient's ear to deliver auditory stimuli that can generate evoked potentials. DSP 1051 can then remove artifacts from the received evoked potential signal and displays the artifact-free waveforms. As may be appreciated, the disclosed embodiments are not limited to embodiments that include stimulus generator 1054 or earphone 1031, or to embodiments that provide stimuli and record evoked responses to such stimuli. In some embodiments, device 1000 can be configured to determine neurological state, disease, dysfunction, or injury without providing stimuli or recording evoked responses to such stimuli.

Additionally, base unit 1042 can contain an internal rechargeable battery 1044 that can be charged during or in between uses by battery charger 1039 connected to an AC outlet 1037.

Consistent with disclosed embodiments, device 1000 can be used for near-patient testing in emergency rooms, ambulatory setting, and other field applications. In some instances, device 1000 can be used in conjunction with CT scan, MRI or other imaging studies to provide complementary or corroborative information about a patient's neurological condition. Device 1000 can be used at point-of-care to more provide an assessment of neurological state, disease, dysfunction, or injury.

Implementations within the scope of the present disclosure can be partially or entirely realized using a non-transitory, computer-readable storage medium (or multiple such non-transitory, computer-readable storage media of one or more types) containing instructions.

The non-transitory, computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

The instructions contained in the non-transitory, computer-readable storage medium can be directly executable or usable to develop executable instructions. For example, such instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

The disclosed embodiments can be implemented using microprocessor or multi-core processors that execute software, integrated circuits, such as GPUs, CPUs, ASICs or FPGAs, or the like. In one or more implementations, integrated circuits can execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

The embodiments may be further described using the following clauses:

1. A subject measurement system comprising: at least one processor; and at least one non-transitory, computer-readable medium containing instructions that, when executed by the at least one processor, cause the subject monitoring system to perform operations comprising: obtaining an electroencephalographic (EEG) dataset of a subject; applying the EEG dataset to a tokenizing model to generate a first token; applying the first token to a sequential machine learning model to generate output features; wherein the tokenizing model, the sequential machine learning model, and a de-tokenizing model are jointly trained to generate an output EEG dataset from an input EEG dataset; applying a classifier input including the output features to a classifier to generate an indication of a neurological state, disease, dysfunction, or injury; and providing the indication of the neurological state, disease, dysfunction, or injury to inform treatment of the subject.

2. The subject measurement system of clause 1, wherein: the sequential machine learning model is an attention-based model, a state-space model, or a recurrent neural network.

3. The subject measurement system of any one of clauses 1 to 2, wherein: at least one of the tokenizing model or the de-tokenizing model is a feed-forward neural network.

4. The subject measurement system of any one of clauses 1 to 3, wherein: the tokenizing model is configured to accept a first number of multiple sequential input observations and output a single output token; or the de-tokenizing model is configured to accept a single input token and output a second number of multiple sequential output results.

5. The subject measurement system of any one of clauses 1 to 4, wherein: the output features comprise or depend upon activation values for one or more layers of the sequential machine learning model.

6. The subject measurement system of any one of clauses 1 to 5, wherein: the classifier input further comprises an indication of a non-EEG physiological measurement, clinical assessment result, or patient demographic information.

7. The subject measurement system of clause 6, wherein: the non-EEG physiological measurement comprises an electrocardiogram, electrooculogram, electromyogram, peripheral electroneurogram, blood or fluid biomarker, or genetic or genomic descriptor.

8. The subject measurement system of any one of clauses 1 to 7, wherein: the EEG dataset is generated using more than 10 ms of EEG signals acquired from the subject.

9. The subject measurement system of any one of clauses 1 to 8, wherein: the classifier comprises at least one of a support vector machine, a linear or logistic regression classifier, a decision tree or random forest model, or a linear or non-linear neural network.

10. The subject measurement system of any one of clauses 1 to 9, wherein: the neurological state, disease, dysfunction, or injury comprises a brain age or a status, classification, or prognosis for epilepsy, concussion, stroke, traumatic brain injury, dementia, or Parkinsons.

11. The subject measurement system of any one of clauses 1 to 10, wherein: the sequential machine learning model is pre-trained to generate textual outputs from textual inputs.

12. A method for training a subject measurement system comprising: training a first combined machine learning model using an electroencephalographic (EEG) pre-training dataset to generate an output sequence of multi-channel EEG observations from an input sequence of multi-channel EEG observations, wherein: the EEG pre-training dataset comprises sets of sequential multi-channel EEG observations; and the first combined machine learning model including: a tokenizing model configured to generate a first token from the input sequence; a sequential machine learning model configured to generate a second token from the first token, generation of the second token including generation of output features; and a de-tokenizing model configured to generate the output sequence from the second token; and training a second combined machine learning model using an EEG training dataset to generate an indication of a neurological state, disease, dysfunction, or injury from an input sequence of multi-channel EEG observations, wherein: the EEG training dataset comprises labeled sets of sequential multi-channel EEG observations, the label for each set indicating a neurological state, disease, dysfunction, or injury; and the second combined machine learning model including: the tokenizing model; the sequential machine learning model; and a classifier configured to generate the indication using a classifier input including the output features.

13. The method of clause 12, wherein: the sequential machine learning model is an attention-based model, a state-space model, or a recurrent neural network.

14. The method of any one of clauses 12 to 13, wherein: at least one of the tokenizing model or the de-tokenizing model is a feed-forward neural network.

15. The method of any one of clauses 12 to 14, wherein: the output features comprise or depend upon activation values for one or more layers of the sequential machine learning model.

16. The method of any one of clauses 12 to 15, wherein: the classifier comprises at least one of a support vector machine, a linear or logistic regression classifier, a decision tree or random forest model, or a linear or non-linear neural network.

17. The method of any one of clauses 12 to 16, wherein: the input sequence corresponds to at least 10 ms of EEG data.

18. The method of any one of clauses 12 to 17, wherein: the neurological state, disease, dysfunction, or injury comprises a brain age or a status, classification, or prognosis for epilepsy, concussion, stroke, traumatic brain injury, dementia, or Parkinsons.

19. A non-transitory computer-readable medium containing instructions that, when executed by at least one processor of a subject measurement system, cause the subject measurement system to perform operations comprising: obtaining an electroencephalographic (EEG) dataset of a subject; applying the EEG dataset to a tokenizing model to generate a first token; applying the first token to a sequential machine learning model to generate output features; wherein the tokenizing model, the sequential machine learning model, and a de-tokenizing model are jointly trained to generate an output EEG dataset from an input EEG dataset; applying the output features to a classifier to generate an indication of a neurological state, disease, dysfunction, or injury; and providing the indication of the neurological state, disease, dysfunction, or injury to inform treatment of the subject.

20. The non-transitory computer-readable medium of clause 19, wherein: the sequential machine learning model is an attention-based model, a state-space model, or a recurrent neural network; at least one of the tokenizing model or the de-tokenizing model is a feed-forward neural network; the output features comprise or depend upon activation values for one or more layers of the sequential machine learning model; and the classifier comprises at least one of a support vector machine, a linear or logistic regression classifier, a decision tree or random forest model, or a linear or non-linear neural network.

21. The non-transitory computer-readable medium of any one of clauses 19 to 20, wherein: the neurological state, disease, dysfunction, or injury comprises a brain age or a status, classification, or prognosis for epilepsy, concussion, stroke, traumatic brain injury, dementia, or Parkinsons.

22. A subject measurement system comprising: at least one processor; and at least one non-transitory, computer-readable medium containing instructions that, when executed by the at least one processor, cause the subject monitoring system to perform operations comprising: generating a first token by applying a subject electroencephalographic (EEG) dataset to at least one tokenizing model; generating a second token corresponding to a subject textual or image input; generating a subject EEG or textual output using the first token and the second token, generation including applying the first token and the second token to a sequential machine learning model, wherein the at least one tokenizing model, the sequential machine learning model, and at least one de-tokenizing model are jointly trained to generate an output training EEG or textual dataset from an input EEG or textual dataset; and providing the second EEG data or textual output.

23. The subject measurement system of clause 22, wherein: the sequential machine learning model is an attention-based model, a state-space model, or a recurrent neural network.

24. The subject measurement system of any one of clauses 22 to 23, wherein: the sequential machine learning model is a pretrained language model; or the sequential machine learning model is frozen during the joint training.

25. The subject measurement system of any one of clauses 22 to 24, wherein: the first token and the second token are in the same vocabulary.

26. The subject measurement system of any one of clauses 22 to 25, wherein: one or more of the at least one tokenizing model or the at least one de-tokenizing model is a feed-forward neural network.

27. The subject measurement system of any one of clauses 22 to 26, wherein: the subject textual or image input comprises: medical data concerning the subject; or instructions for processing the EEG dataset.

28. The subject measurement system of clause 27, wherein: the medical data of the subject comprises brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data.

29. The subject measurement system of clause 27, wherein: the instructions specify a temporal or signal-source subset of the EEG dataset; or the instructions specify the contents of the output training EEG or textual dataset.

30. The subject measurement system of any one of clauses 22 to 29, wherein: the at least one tokenizing model includes a tokenizing model configured to accept EEG data or a tokenizing model configured to accept textual data; or the at least one de-tokenizing model includes a de-tokenizing model configured to output EEG data or a de-tokenizing model configured to output textual data.

31. The subject measurement system of any one of clauses 22 to 30, wherein: the at least one tokenizing model is configured to accept a first number of multiple sequential input observations and output a single output token; or the at least one de-tokenizing model is configured to accept a single input token and output a second number of multiple sequential output results.

32. The subject measurement system of any one of clauses 22 to 31, wherein: providing the second EEG data or textual output comprises providing a textual indication of a neurological state, disease, dysfunction, or injury of the subject.

33. The subject measurement system of clause 32, wherein: the neurological state, disease, dysfunction, or injury of the subject comprises a brain age or a status, classification, or prognosis for epilepsy, concussion, stroke, traumatic brain injury, dementia, or Parkinsons.

34. A method for training a subject measurement system comprising: training a first combined machine learning model using an electroencephalographic (EEG) and textual pre-training dataset to generate an output sequence of multi-channel EEG observations and textual values from an input sequence of multi-channel EEG observations and textual or image input data, wherein: the EEG and textual pre-training dataset comprises: sequential multi-channel EEG observations for at least one subject, textual or image input data including instructions for processing the sequential multi-channel EEG observations, and output textual data including indications of neurological states, diseases, dysfunctions, or injuries associated with the multi-channel EEG observations; and the first combined machine learning model including: a tokenizing model configured to generate a first token from the input sequence; a sequential machine learning model configured to generate a second token from the first token; and a de-tokenizing model configured to generate the output sequence from the second token; and training the first combined machine learning model comprises updating at least one of the tokenizing model or the detokenizing model based on a comparison of the input sequence and the output sequence.

35. The method of clause 34, wherein: the sequential machine learning model is an attention-based model, a state-space model, or a recurrent neural network.

36. The method of any one of clauses 34 to 35, wherein: at least one of the tokenizing model or the de-tokenizing model is a feed-forward neural network.

37. The method of any one of clauses 34 to 36, wherein: the neurological state, disease, dysfunction, or injury comprises a brain age or a status, classification, or prognosis for epilepsy, concussion, stroke, traumatic brain injury, dementia, or Parkinsons.

38. The method of any one of clauses 34 to 37, wherein: textual or image input data further includes medical data concerning the subject.

39. The method of clause 38, wherein: the medical data of the subject comprises brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data.

40. The method of any one of clauses 34 to 39, wherein: the instructions specify a temporal or signal-source subset of the EEG dataset; or the instructions specify the contents of the output training EEG or textual dataset.

41. A non-transitory computer-readable medium containing instructions that, when executed by at least one processor of a subject measurement system, cause the subject measurement system to perform operations comprising: generating a first token by applying a subject electroencephalographic (EEG) dataset to a tokenizing model; generating a second token corresponding to a subject textual input; generating a subject EEG or textual output using the first token and the second token, generation including applying the first token and the second token to a sequential machine learning model, wherein the tokenizing model, the sequential machine learning model, and a de-tokenizing model are jointly trained to generate an output training EEG or textual dataset from an input EEG or textual dataset; and providing the second EEG data or textual output.

42. A subject measurement system comprising: at least one processor; and at least one non-transitory, computer-readable medium containing instructions that, when executed by the at least one processor, cause the subject measurement system to perform operations comprising: generating a classifier or textual output using at least one timeseries dataset acquired from a subject and a second dataset concerning the subject, a second modality of the second dataset differing from at least one first modality of the at least one timeseries dataset, generation comprising: generating at least one first token, at least in part by applying the at least one timeseries dataset to at least one corresponding tokenizing model; generating a second token using the second dataset; generating a classifier or textual output at least in part by applying the at least one first token and the second token to a sequential machine learning model; and wherein the at least one corresponding tokenizing model, the sequential machine learning model, and at least one de-tokenizing model are jointly trained to generate at least one predicted output timeseries dataset in the at least one first modality from at least one input timeseries dataset in the at least one first modality; and providing the classifier or textual output.

43. The subject measurement system of clause 42, wherein: the sequential machine learning model is an attention-based model, a state-space model, or a recurrent neural network.

44. The subject measurement system of any one of clauses 42 to 43, wherein: the sequential machine learning model is a pretrained language model; or the sequential machine learning model is frozen during the joint training.

45. The subject measurement system of any one of clauses 42 to 44, wherein: the at least one first token and the second token are in the same vocabulary.

46. The subject measurement system of any one of clauses 42 to 45, wherein: at least one of the tokenizing model or the de-tokenizing model is a feed-forward neural network.

47. The subject measurement system of any one of clauses 42 to 46, wherein: the second dataset comprises medical data concerning the subject.

48. The subject measurement system of clause 47, wherein: the medical data concerning the subject comprises brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data.

49. The subject measurement system of any one of clauses 42 to 48, wherein: generating the classifier or textual output further comprises applying textual instructions to the sequential machine learning model.

50. The subject measurement system of clause 49, wherein: the operations specify generating a textual output and the textual instructions specify the contents of the textual output; the textual instructions specify a temporal or signal-source portion of the at least one timeseries dataset; or the textual instructions specify a modality of the at least one first token or a modality of the second token.

51. The subject measurement system of any one of clauses 42 to 50, wherein: the at least one timeseries dataset comprises an EEG timeseries dataset and a speech timeseries dataset.

52. The subject measurement system of any one of clauses 42 to 51, wherein: the at least one tokenizing model is configured to accept multiple timeseries observations and output a single output token; or the at least one de-tokenizing model is configured to accept a single input token and output a second number of multiple sequential output results.

53. The subject measurement system of any one of clauses 42 to 52, wherein: providing the classifier or textual output comprises providing a textual indication of a neurological state, disease, dysfunction, or injury of the subject.

54. The subject measurement system of clause 53, wherein: the neurological state, disease, dysfunction, or injury of the subject comprises a brain age or a status, classification, or prognosis for epilepsy, concussion, stroke, traumatic brain injury, dementia, or Parkinsons.

55. A training system, comprising: at least one processor; and at least one non-transitory, computer-readable medium containing instructions that, when executed by the at least one processor, cause the training system to perform operations comprising: training at least one tokenizing model and at least one de-tokenizing model to generate at least one predicted output timeseries dataset in at least one first modality from at least one first input timeseries dataset in the at least one first modality using a sequential machine learning model, training the at least one tokenizing model and the at least one de-tokenizing model comprising: generating at least one first token, at least in part by applying the at least one first input timeseries dataset to the at least one tokenizing model; generating a first output sequence, at least in part by applying a first input sequence including the at least one first token to the sequential machine learning model; extracting at least one first output token from the first output sequence; and generating the least one predicted output timeseries dataset, at least in part by applying the at least one first output token to the at least one de-tokenizing model; and storing the at least one trained tokenizing model and the at least one trained de-tokenizing model.

56. The training system of clause 55, wherein the operations further comprise: training a classifier to generate classifications from at least one second input timeseries dataset in the at least one first modality using corresponding label data, the at least one trained tokenizing model, and the sequential machine learning model, training the classifier comprising: generating at least one second token, at least in part by applying the at least one second input timeseries dataset to the at least one trained tokenizing model; generating a classifier input, at least in part by applying a second input sequence including the at least one second token to the sequential machine learning model; generating a classifier output, at least in part by applying the classifier input to classifier; and updating the classifier based on a comparison of the classifier output and the corresponding label data.

57. The training system of clause 56, wherein: the classifier input comprises internal state information of the sequential machine learning model or a function of the internal state information of the sequential machine learning model; or the classifier input comprises an output of the sequential machine learning model or a function of the output of the sequential machine learning model.

58. The training system of any one of clauses 56 to 57, wherein: training the classifier further comprises generating at least one third token using a third dataset comprising medical data concerning the subject, the medical data concerning the subject comprising brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data; and the second input sequence further includes the at least one third token.

59. The training system of any one of clauses 56 to 58, wherein: the second input sequence further includes at least one textual instruction or modality delimiter.

60. The training system of any one of clauses 55 to 59, wherein: the at least one first input timeseries dataset comprises an EEG timeseries dataset and a speech timeseries dataset.

61. The training system of any one of clauses 55 to 60, wherein: the at least one first output token is extracted from the first output sequence based on modality delimiters in the first output sequence that specify the at least one de-tokenizing model.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A subject measurement system comprising:
at least one processor; and
at least one non-transitory, computer-readable medium containing instructions that, when executed by the at least one processor, cause the subject measurement system to perform operations comprising:
generating a classifier or textual output using at least one timeseries dataset acquired from a subject and a second dataset concerning the subject, a second modality of the second dataset differing from at least one first modality of the at least one timeseries dataset, generation comprising:
generating at least one first token, at least in part by applying the at least one timeseries dataset to at least one corresponding tokenizing model;
generating a second token using the second dataset;
generating a classifier or textual output at least in part by applying the at least one first token and the second token to a sequential machine learning model; and
wherein the at least one corresponding tokenizing model, the sequential machine learning model, and at least one de-tokenizing model are jointly trained to generate at least one predicted output timeseries dataset in the at least one first modality from at least one input timeseries dataset in the at least one first modality; and
providing the classifier or textual output.

2. The subject measurement system of claim 1, wherein:
the sequential machine learning model is an attention-based model, a state-space model, or a recurrent neural network.

3. The subject measurement system of claim 1, wherein:
the sequential machine learning model is a pretrained language model; or
the sequential machine learning model is frozen during the joint training.

4. The subject measurement system of claim 1, wherein:
the at least one first token and the second token are in the same vocabulary.

5. The subject measurement system of claim 1, wherein:
at least one of the tokenizing model or the de-tokenizing model is a feed-forward neural network.

6. The subject measurement system of claim 1, wherein:
the second dataset comprises medical data concerning the subject.

7. The subject measurement system of claim 6, wherein:
the medical data concerning the subject comprises brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data.

8. The subject measurement system of claim 1, wherein:
generating the classifier or textual output further comprises applying textual instructions to the sequential machine learning model.

9. The subject measurement system of claim 8, wherein:
the operations specify generating a textual output and the textual instructions specify the contents of the textual output;
the textual instructions specify a temporal or signal-source portion of the at least one timeseries dataset; or
the textual instructions specify a modality of the at least one first token or a modality of the second token.

10. The subject measurement system of claim 1, wherein:
the at least one timeseries dataset comprises an EEG timeseries dataset and a speech timeseries dataset.

11. The subject measurement system of claim 1, wherein:
the at least one tokenizing model is configured to accept multiple timeseries observations and output a single output token; or
the de-tokenizing model is configured to accept a single input token and output a second number of multiple sequential output results.

12. The subject measurement system of claim 1, wherein:
providing the classifier or textual output comprises providing a textual indication of a neurological state, disease, dysfunction, or injury of the subject.

13. The subject measurement system of claim 12, wherein:
the neurological state, disease, dysfunction, or injury of the subject comprises a brain age or a status, classification, or prognosis for epilepsy, concussion, stroke, traumatic brain injury, dementia, or Parkinsons.

14. A training system, comprising:
at least one processor; and
at least one non-transitory, computer-readable medium containing instructions that, when executed by the at least one processor, cause the training system to perform operations comprising:
training at least one tokenizing model and at least one de-tokenizing model to generate at least one predicted output timeseries dataset in at least one first modality from at least one first input timeseries dataset in the at least one first modality using a sequential machine learning model, training the at least one tokenizing model and the at least one de-tokenizing model comprising:
generating at least one first token, at least in part by applying the at least one first input timeseries dataset to the at least one tokenizing model;
generating a first output sequence, at least in part by applying a first input sequence including the at least one first token to the sequential machine learning model;
extracting at least one first output token from the first output sequence; and
generating the least one predicted output timeseries dataset, at least in part by applying the at least one first output token to the at least one de-tokenizing model; and
storing the at least one trained tokenizing model and the at least one trained de-tokenizing model.

15. The training system of claim 14, wherein the operations further comprise:
training a classifier to generate classifications from at least one second input timeseries dataset in the at least one first modality using corresponding label data, the at least one trained tokenizing model, and the sequential machine learning model, training the classifier comprising:
generating at least one second token, at least in part by applying the at least one second input timeseries dataset to the at least one trained tokenizing model;
generating a classifier input, at least in part by applying a second input sequence including the at least one second token to the sequential machine learning model;
generating a classifier output, at least in part by applying the classifier input to classifier; and
updating the classifier based on a comparison of the classifier output and the corresponding label data.

16. The training system of claim 15, wherein:
the classifier input comprises internal state information of the sequential machine learning model or a function of the internal state information of the sequential machine learning model; or
the classifier input comprises an output of the sequential machine learning model or a function of the output of the sequential machine learning model.

17. The training system of claim 15, wherein:
training the classifier further comprises generating at least one third token using a third dataset comprising medical data concerning the subject, the medical data concerning the subject comprising brain imaging data, heart rate variability, pulse oxygenation, respiration, electrocardiogram, electrooculogram, electromyogram, peripheral electroneurogram, blood or fluid biomarker, genetic or genomic descriptor data, or clinical assessment data; and
the second input sequence further includes the at least one third token.

18. The training system of claim 15, wherein:
the second input sequence further includes at least one textual instruction or modality delimiter.

19. The training system of claim 14, wherein:
the at least one first input timeseries dataset comprises an EEG timeseries dataset and a speech timeseries dataset.

20. The training system of claim 14, wherein:
the at least one first output token is extracted from the first output sequence based on modality delimiters in the first output sequence that specify the at least one de-tokenizing model.
